(19) United States
(12) Patent Application Publication
(10) Pub. No.: US 2026/0049126 A1
(43) Pub. Date: Feb. 19, 2026

(54) FOLLISTATIN-FC FUSION PROTEINS

(71) Applicant: UCB BIOPHARMA SRL, Brussels (BE)

(72) Inventors: James Robert Chaves Birtley, Slough (GB); Lara Kevorkian, Slough (GB); David James McMillan, Slough, Berkshire (GB)

(21) Appl. No.: 18/875,036

(22) PCT Filed: Jun. 14, 2023
(86) PCT No.: PCT/EP2023/065930

§ 371 Date: Dec. 13, 2024

(30) Foreign Application Priority Data
Jun. 15, 2022 (WO) PCT/EP2022/066334

(51) Int. Cl. C07K 16/18; A61K 38/00; C07K 14/47; C12N 15/62; C12N 15/85

(52) U.S. Cl. CPC C07K 16/18; A61K 38/00; C07K 14/4703; C12N 15/62; C12N 15/85; C07K 2317/24; C07K 2317/54; C07K 2317/55; C07K 2317/565; C07K 2317/92; C07K 2319/00

(57) ABSTRACT

The invention relates to the field of fusion proteins and in particular to fusion proteins comprising a follistatin moiety. The invention also relates to methods of making said fusion proteins, together with pharmaceutical formulations comprising said fusion proteins.

### CROSS-REFERENCE TO RELATED APPLICATIONS

This application is a U.S. national phase of International Patent Application No. PCT/EP2023/065930, filed Jun. 14, 2023, which claims the benefit of priority of International Patent Application No. PCT/EP2022/066334, filed on Jun. 15, 2022, the contents of which are incorporated by reference in its entirety.

### INCORPORATION BY REFERENCE

This application contains, as a separate part of disclosure, a sequence listing in computer-readable form (filename: 70815_SubSeqListing.xml; Size: 106,963 bytes; Created: Dec. 26, 2024), which is incorporated by reference in its entirety.

### FIELD OF THE INVENTION

The invention relates to the field of fusion proteins and in particular to fusion proteins comprising a follistatin moiety. The invention also relates to methods of making said fusion proteins, together with pharmaceutical formulations comprising said fusion proteins.

### BACKGROUND OF THE INVENTION

Follistatin is an autocrine glycoprotein which has as its primary function the binding and neutralisation of members of the TGF-beta superfamily and in particular Activin A, Activin B, GDF8 (myostatin) and GDF11. It is known to exist in several different forms, including a 315-amino acid polypeptide (designated FST315); and a 288-amino acid polypeptide (designated FST288), as shown in FIG. 1. Both FST315 and FST288 have high affinity for activins (Activin A & Activin B) as well as for myostatin (GDF8). In particular, follistatin can bind to and inhibit myostatin, which is a negative regulator of skeletal muscle mass.

Follistatin has been shown to be a potential therapeutic protein in certain conditions, including in treatment of muscle disorders such as muscular dystrophy (WO2015/187977 and WO2017/152090). However, the use of follistatin in therapy has run into a number of obstacles, based primarily on the difficulty of expressing follistatin in vitro and on the low stability/short half-life of follistatin in vivo. There have been attempts to overcome these obstacles, and both WO2015/187977 and WO2017/152090 discuss the use of fusion proteins comprising a follistatin polypeptide fused to the Fc portion of an immunoglobulin.

There remains a need for follistatin peptides and fusion proteins which can be more readily expressed in vitro and which have an improved half-life or other beneficial effects in vivo.

### SUMMARY OF THE INVENTION

In a first aspect, the invention provides a fusion protein comprising: a follistatin moiety, an antibody moiety, and optionally a linker between the follistatin moiety and the antibody moiety.

In some embodiments, the antibody moiety binds albumin (such as serum albumin (SA)) and the follistatin moiety comprises or is a naturally occurring protein, a functional fragment thereof and/or a functional variant thereof. In some examples, the follistatin moiety is selected from: a. SEQ ID NO: 1, b. SEQ ID NO: 2, c. SEQ ID NO: 3, d. SEQ ID NO: 4, e. any protein comprising amino acid residues comprising between 289 and 314 residues of any one of SEQ ID NOs. 1 to 4; or f. a sequence having at least 95%, at least 96%, at least 97%, at least 98% or at least 99% sequence identity to any one of SEQ ID NOs: 1 to 4.

In some specific examples the fusion protein comprises or consists of:
(a) i. an FST315 polypeptide defined by SEQ ID NO: 1, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315 polypeptide; and
iii. a Fab light chain defined by SEQ ID NO: 6, or a sequence having at least 95%, at least 96%, at least 97%, at least 98% or at least 99% sequence identity thereto;
(b) i. an FST288 polypeptide defined by SEQ ID NO: 2, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288 polypeptide; and
iii. a Fab light chain defined by SEQ ID NO: 6, or a sequence having at least 95%, at least 96%, at least 97%, at least 98% or at least 99% sequence identity thereto;
(c) i. an FST315HBM polypeptide defined by SEQ ID NO: 3, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315HBM polypeptide; and
iii. a Fab light chain defined by SEQ ID NO: 6, or a sequence having at least 95%, at least 96%, at least 97%, at least 98% or at least 99% sequence identity thereto;
(d) i. an FST288HBM polypeptide defined by SEQ ID NO: 4, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288HBM polypeptide; and
iii. a Fab light chain defined by SEQ ID NO: 6, or a sequence having at least 95%, at least 96%, at least 97%, at least 98% or at least 99% sequence identity thereto;
(e) i. an FST315 polypeptide defined by SEQ ID NO: 1, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(f) i. an FST288 polypeptide defined by SEQ ID NO: 2, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(g) i. an FST315 polypeptide variant defined by SEQ ID NO: 3 (FST315HBM) or SEQ ID NO: 22, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(h) i. an FST288 polypeptide variant defined by SEQ ID NO: 4 (FST288HBM) or SEQ ID NO:25, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(i) SEQ ID NO: 8, 9, 10, 11, 24, 25, 26, 27, 32, 33, 34 or 35 or a sequence having at least 95%, at least 96%, at least 97%, at least 98% or at least 99% sequence identity thereto and a Fab light chain defined by SEQ ID NO: 6, or a sequence having at least 95%, at least 96%, at least 97%, at least 98% or at least 99% sequence identity thereto;
(j) SEQ ID NO: 8, 9, 10, 11, 24, 25, 26, 27, 32, 33, 34 or 35 and a Fab light chain defined by SEQ ID NO: 6; or
(k) a functional variant or fragment of any one of (a) to (j).

In another aspect, the invention relates to i) one or more isolated polynucleotide(s) encoding the fusion protein of the invention; ii) one or more cloning or expression vector(s) comprising one or more polynucleotides of the invention; as well as iii) a host cell comprising one or more polynucleotide(s) according to the invention or one or more expression vector(s) according to the invention.

In yet another aspect, the invention provides a process for the production of a fusion protein according to the invention, comprising culturing the host cell according to the invention under suitable conditions for producing the fusion protein and isolating the fusion protein.

In a further aspect, the invention relates to a pharmaceutical composition comprising the fusion protein according to the invention and one or more pharmaceutically acceptable carriers, excipients or diluents.

In an additional aspect, the fusion proteins according to the invention or the pharmaceutical compositions according to the invention are for use in therapy.

### DESCRIPTION OF THE FIGURES

FIG. 1: A schematic illustrating domain organisation of two mature follistatin moieties (FST288 and FST315; i.e. without their N-terminal secretion signal sequence) and how an exemplary pair of FST288 molecules form a complex with an activin disulphide-linked homodimer. FST288 and FST 315 both have four domains in common which includes an N-terminal domain, FSD1, FSD2 and FSD3. FST315 can also bind activin and has an extra C-terminal domain (residues 289-315).

FIG. 2: (A) Relative expression levels of follistatin fusion proteins were determined by protein-G HPLC analysis of harvested CHO supernatants. Values are normalised relative to the FST-Fc expression level. N-Fab-FST=Fab antibody moiety fused to the N-terminal portion of FST; FST-Fab-C=Fab antibody moiety fused to the C-terminal portion of FST; FST-Fc=Fc antibody moiety fused to the C-terminal portion of FST; FST-ScFv=ScFv antibody moiety fused to the C-terminal portion of FST. (B) Relative expression levels of FST288, FST315, FSTWT and FST-HBM (alternatively named HBSM) with a C-terminal fusion partner (respectively named 288 fusions, 315 fusions, WT fusions and HBSM fusions in the Figure). Values were normalised to the expression of the 288 fusions. (C) Total expression values of monomer, values are normalised relative to the FST-Fc expression level, n=25. Same legend as for FIG. 2(A).

FIG. 3: (A) Pharmacokinetic profile of FST315WT, FST315-Fab and FST315HBM-Fab, dosed into mice (n=3 mice per group) at 10 mg/kg via an IV administration route and serum samples were monitored for 7 days; (B) Pharmacokinetic profile of FST288WT, FST288-Fab and FST288HBM-Fab dosed into mice (n=3 mice per group) at 10 mg/kg via an IV administration route and serum samples were monitored for 7 days. The pharmacokinetic parameters derived from all the follistatin moieties of the study are summarized in Table 1.

FIG. 4: Functional inhibitory effects of (A) FST315HBM-Fab and FST315WT, (B) FST288HBM-Fab and FST288WT in a reporter gene cell assay stimulated with either of the 4 main FST ligands—Activin A, Activin B, GDF8 (Myostatin) and GDF11. All follistatin fusion proteins, dose-dependently inhibited the signal with all 4 ligands and a representative graph shows the different potency profiles of the 4 FST moieties against the different ligands, plotting percentage inhibition versus FST concentration. These experiments have been performed multiple times—FST315HBM-Fab, n=7, FST315WT, n=4, FST288HBM-Fab, n=4 and FST288WT, n=4 and all the data is summarized in Table 3, where the data is presented as IC50 and ranges in nM for the 4 ligands.

FIG. 5: SDS-PAGE Analysis of Protein A Elution and Strip pools, Lane (1) Molecular weight marker, lane (2) Acidic Elution pool, lane (3) Acidic Strip post acidic elution, lane (4) Alkali Elution pool, lane (5) Acidic Strip post alkali elution where (M)=Full length Fst-Fab monomer (F)=Fab.

FIG. 6: Analytical Size Exclusion. (A) Acidic Elution pool (B) Alkali Elution pool (C) Acidic Strip post acidic elution (D) Acidic Strip post alkali elution where (M)=Full length Fst-Fab monomer and (F)=Fab.

FIG. 7: Histogram showing relative expression levels (g/L) of 4 cell clones assessed in fed batch processes. Clones assessed in two different media (media A and B) and two different feeds conditions (FB1 and FB2). Total protein concentration determined by CH1 HPLC.

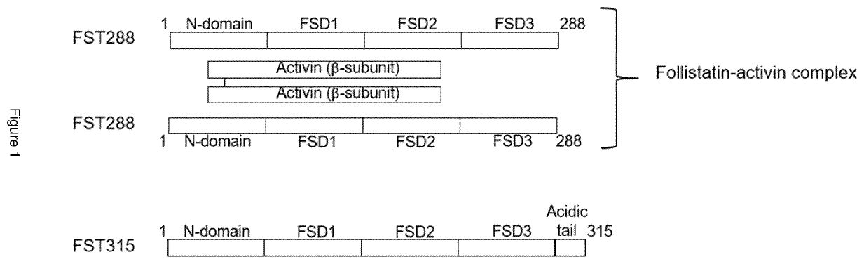

### DETAILED DESCRIPTION OF THE INVENTION

Technical terms are used by their common sense unless indicated otherwise. If a specific meaning is conveyed to certain terms, definitions of terms will be given in the context of which the terms are used.

Where an indefinite or definite article is used when referring to a singular noun, e.g. “a”, “an” or “the”, this includes a plural of that noun unless something else is specifically stated. As used here, the term “comprising” does not exclude other elements. For the purposes of the present disclosure, the term “consisting of” is considered to be a preferred embodiment of the term “comprising of”. The terms “Follistatin” or “FST”, as used herein, refer to an autocrine glycoprotein (UniProt reference: P19883) which is a known inhibitor of Activins A and B. Follistatin also binds with lower affinity to GDF11, GDF8 (Myostatin), BMPs 2, 4, 6, 7, 11, and 15. There are two main alternatively spliced forms of human Follistatin: a shorter form (FST288, 31.6 kDa) which is cell bound and a longer circulating form (FST315, 34.8 kDa). The FST315 is defined according to SEQ ID NO: 1 and the FST288 is defined according to SEQ ID NO: 2 (SEQ ID NOs: 1 and 2 are both mature forms, lacking N-terminal secretion signal peptide). The FST315 and FST288, have four domains stabilised by network of disulphide bonds (18 in total), two N-linked glycosylation sites and one heparin binding site. FST315 has an additional 27 amino acids domain (acidic rich) at its C-terminus termed the acidic tail. Also encompassed by the terms are functional fragments and/or functional variants thereof, such as those disclosed in Sidis et al., 2005. If followed by a number, e.g. FST288, this indicates that the protein is the 288 form of follistatin (starting at residue 1 of the mature form). If followed by a number and letters, e.g. FST315HBM, this indicates the heparin-binding mutant (HBM) form as well as the type of variant (here the 315 form of follistatin, starting at residue 1 of the mature form, and including Alanine mutations at residues K76, K81 and K82). Activins are dimeric polypeptide growth factors and belong to the TGF-beta superfamily. Activins can stimulate hormone production in ovarian and placental cells, support neuronal cell survival, and influence cell-cycle progress positively or negatively, depending on cell type. In several tissues, activin signalling is antagonized by its related heterodimer, inhibin. For example, during the release of follicle-stimulating hormone (FSH) from the pituitary, activin promotes FSH secretion and synthesis, while inhibin prevents FSH secretion and synthesis. Activin has also been implicated as a negative regulator of muscle mass and function, and activin antagonists can promote muscle growth or counteract muscle loss in vivo.

The term “antibody” as used herein includes, but is not limited to, monoclonal antibodies, polyclonal antibodies and recombinant antibodies that are generated by recombinant technologies as known in the art. The term “antibody” as used herein includes antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” refers to both glycosylated and aglycosylated antibodies. Furthermore, the term “antibody moiety” as used herein may refer to full-length antibodies, but more generally is intended to reference antibody fragments, and more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). The term “Fab” refers to as used herein refers to an antibody fragment comprising a light chain fragment comprising a VL (variable light) domain and a constant domain of a light chain (CL), and a VH (variable heavy) domain and a first constant domain (CH1) of a heavy chain.

The term “Fab′” as employed herein is similar to a Fab, wherein the Fab portion is replaced by a Fab′. The format may be provided as a PEGylated version thereof. Dimers of a Fab′ according to the present disclosure create a F(ab′)2 where, for example, dimerization may be through the hinge. The term “Fv” refers to two variable domains of full-length antibodies, for example co-operative variable domains, such as a cognate pair or affinity matured variable domains, i.e. a VH and VL pair.

The term “single chain variable fragment” or “scFv” as employed herein refers to a single chain variable fragment which is stabilised by a peptide linker between the VH and VL variable domains. The term “single domain antibody” as used herein refers to an antibody fragment consisting of a single monomeric variable domain. Examples of single domain antibodies include VH or VL or VHH or V-NAR.

As used herein, the term “affinity” refers to the strength of all noncovalent interactions between a protein or a fragment thereof and its receptor (if the protein of interest is a ligand) or its ligand (if the protein of interest is a receptor). Unless indicated otherwise, as used herein, the term “binding affinity” refers to intrinsic binding affinity which reflects a 1:1 interaction between members of a binding pair (e.g., a receptor and its ligand). The affinity of a molecule for its binding partner can be generally represented by the dissociation constant (KD). Affinity can be measured by common methods known in the art, including those described herein.

The term “specific” as employed herein in the context of antibodies and antigen-binding fragments is intended to refer to an antibody that only recognizes the antigen to which it is specific or an antibody that has significantly higher binding affinity to the antigen to which it is specific compared to binding to antigens to which it is non-specific, for example at least 5, 6, 7, 8, 9, 10 times higher binding affinity.

The terms “albumin”, “serum albumin” or “SA” refer to an abundant globular proteins in both vascular and extravascular compartments. The human form of serum albumin (HSA) is known under reference P02768, whereas the mouse serum equivalent is referenced as P07724.

The term “chimeric” refers to antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences.

“Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. Unless indicated otherwise, HVR residues (CDR residues) and other residues in the variable domain (e.g., FR residues) are numbered herein according to Kabat.

The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody.

The term “functional variant” as used herein refers to an amino acid sequence which has been modified relative to a reference sequence but which retains at least one biological function of said reference sequence. For example, a functional variant of FST retains at least one biological activity of the reference FST protein, such as binding and inhibition of Activins A and B.

As used herein, the term “sequence identity” or “identity” refers to the number of matches (identical nucleic acid or amino acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Alignment for purposes of determining percent nucleic acid or amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid or amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The term “isolated” means, throughout this specification, that the antibody, antigen-binding fragment, polypeptide or polynucleotide, as the case may be, exists in a physical milieu distinct from that in which it may occur in nature. The term “isolated” nucleic acid refers to a nucleic acid molecule that has been isolated from its natural environment or that has been synthetically created. An isolated nucleic acid may comprise synthetic DNA, for instance produced by chemical processing, cDNA, genomic DNA or any combination thereof.

The terms “nucleic acid” and “polynucleotide” or “nucleotide sequence” may be used interchangeably to refer to any molecule composed of or comprising monomeric nucleotides. A nucleic acid may be an oligonucleotide or a polynucleotide. A nucleotide sequence may be a DNA or RNA.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, and/or vehicle with which the therapeutic agent is administered.

The term “therapeutically effective amount” refers to the amount that, when administered to a subject for treating a disease, is sufficient to produce such treatment for the disease.

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject, i.e. a human, which may be predisposed to the disease but has not yet been diagnosed as having it; (b) inhibiting the disease, i.e., arresting its development; and (c) relieving the disease, i.e., causing regression of the disease.

The present invention will now be described with respect to particular non-limiting aspects and embodiments thereof and with reference to certain figures and examples.

The present invention addresses the need for improved follistatin peptides and fusion proteins by providing new follistatin fusion proteins which incorporate an antigen-binding antibody moiety (such as an antigen-binding moiety), said fusion proteins being more readily expressed in vitro and having an improved half-life or other beneficial effects in vivo.

The present invention is based on the surprising finding from the inventors that follistatin-based fusion proteins which incorporate an antigen-binding moiety exhibit superior protein expression and higher monomeric fraction yield than previously-known follistatin-based fusion proteins which comprise an Fc moiety. In particular, the fusion proteins of the invention have been shown to have an expression level that is at least 1.5 times greater than FST-Fc fusion proteins. Not only the fusion proteins of the invention have a higher relative expression when compared to the FST-Fc fusion protein, but they also result in a much higher yield of the monomeric fraction, i.e. correctly-folded, usable fusion protein (at least 1.5 times greater than an FST-Fc fusion).

The main object/aspect of the present invention is a fusion protein comprising or consisting of: a. a follistatin moiety, b. an antibody moiety, and optionally c. a linker between the follistatin moiety and the antibody moiety.

In the context of the invention as a whole, the follistatin moiety comprises or is a naturally occurring follistatin protein. It is preferably a mature form thereof, i.e. lacking the N-secretion signal sequence as this sequence is needed only for the production/secretion from a cell. Alternatively, it is a functional fragment thereof. Said follistatin moiety is for instance the FST288 protein (SEQ ID NO. 2) or the FST315 protein (SEQ ID NO. 1). Any intermediate forms thereof, e.g. any follistatin moieties comprising between 289 and 314 residues of any one of SEQ ID NOs. 1 to 4, can also be used, as long as they are functional, i.e. that they retain at least one biological activity of FST. Although none limiting, preferably any intermediate forms of the follistatin moieties start at residue 1 of SEQ ID NO:1. As none limiting examples, the functional FST fragment can be FST291 (i.e. comprising residues 1 to 291 of SEQ ID No. 1) or FST303 (i.e. comprising residues 1 to 303 of SEQ ID No. 1). In another alternative, the follistatin moiety according to the invention (i.e. naturally occurring or functional fragment thereof) is a functional variant, e.g. it can have one or more mutations, such as mutations in the heparin binding site (HBS). As none limiting example, the one or more mutation sites can be selected from K76, K81 and/or K82 numbered relative to SEQ ID NO: 1 (see sequences 22 and 25 as examples). The one or more mutations may comprise Alanine (A) in place of Lysine (K) (resulting in mutations selected from K76A, K81A and/or K82A). As a further none limiting example, a heparin binding mutant (“HBM”, alternatively herein named “(HBM)” or “HBSM”) can be used, e.g. FST288HBM (SEQ ID NO: 4), FST291 HBM, FST303HBM or FST315HBM (SEQ ID NO: 3), wherein said mutant comprises the triple mutations K76A, K81A and K82A.

In particular, the fusion proteins according to the invention comprise a follistatin moiety: a) comprising or consisting of SEQ ID NO: 1, SEQ ID NO: 2, SEQ ID NO: 3, SEQ ID NO: 4, b) comprising or consisting of between 289 and 314 residues of any one of SEQ ID NOs. 1 to 4, or c) comprising or consisting of an amino acid sequence having at least 95%, at least 96%, at least 97%, at least 98% or at least 99% sequence identity to any one of SEQ ID NOs: 1 to 4.

Without wishing to be bound by any theory, the present inventors hypothesise that a fusion protein of the invention exhibits greater stability and/or efficacy in vivo than wild-type follistatin because the antibody moiety in the fusion protein of the invention is capable of binding e.g. to free HSA in the subject, thereby extending the half-life of the fusion protein.

Therefore, in the context of the invention as a whole the antibody moiety preferably binds albumin, and preferably binds serum albumin (SA), such as mouse, rat, cyno or human SA. More preferably, the antibody moiety binds human HSA. Said antibody moiety can be a chimeric, humanized or human antibody moiety. Preferably, the antibody moiety of the fusion protein of the invention is an antigen-binding fragment of an antibody (alternatively herein called antigen-binding moiety). Preferably such antigen-binding moiety is selected from a Fab, a Fab′, or a F(ab′)2. In an alternative, the antibody moiety of the fusion protein of the invention is selected from a Fab, a Fab′, or a F(ab′)2 and comprises a human VH3 domain which is capable of binding protein A.

In one embodiment, the antibody moiety of the fusion protein of the invention comprises a light chain variable region comprising a CDR-L1 comprising SEQ ID NO: 13; a CDR-L2 comprising SEQ ID NO: 14 and a CDR-L3 comprising SEQ ID NO: 15; and a heavy chain variable region comprising a CDR-H1 comprising SEQ ID NO: 16; a CDR-H2 comprising SEQ ID NO: 17 and/or a CDR-H3 comprising SEQ ID NO: 18.

In an alternative embodiment, the antibody moiety of the fusion protein of the invention comprises a heavy chain variable region comprising or consisting of SEQ ID NO: 5, or a sequence having at least 95%, at least 96%, at least 97%, at least 98% or at least 99% sequence identity thereto; and a light chain variable region comprising or consisting of SEQ ID NO: 6, or a sequence having at least 95%, at least 96%, at least 97%, at least 98% or at least 99% sequence identity thereto. SEQ ID NO: 5 and SEQ ID NO: 6 represent the heavy and light variable chains of an anti-albumin antibody designated “CA645” (as disclosed in WO2013/068571). As confirmed in the Examples below, the present inventors have surprisingly found that an FST-Fab fusion protein comprising the heavy and light variable chains of SEQ ID NO: 5 and SEQ ID NO: 6, respectively, are easier to produce and purify.

In the context of the invention as a whole, the fusion proteins optionally comprise a linker between the follistatin moiety and the antibody moiety. When the linker is present as none limiting examples it can be selected from the group consisting of: SGGGGS (SEQ ID NO: 7), SGGGGSSGGGGS (SEQ ID NO: 19), GGGGS (SEQ ID NO: 20) and GGGGSGGGGS (SEQ ID NO: 21).

It will be understood that when creating a fusion protein, there are two options for fusing any moieties to each other: C-terminal fusion or N-terminal fusion. As shown in the Examples below, the present inventors have surprisingly found that fusing the antibody moiety in C-terminal of the follistatin moiety resulted in yet further improved expression of the resulting fusion protein, by comparison with any other type of fusions, such as the antibody moiety fused to the N-terminal portion of the follistatin moiety. In particular, the C-terminal fusion proteins of the invention (i.e. an antibody moiety fused in C-terminal of the follistatin, either directly or via a linker) exhibit superior expression and higher yield of monomeric protein by comparison with the known Fc-based follistatin fusion proteins. However, although the C-terminal fusion proteins resulted in the highest expression level, antibody moiety fused to the N-terminal of follistatin could be considered by the skilled persons as they result for instance in about 1.5 higher expression level compared to Fc-based follistatin fusion proteins.

Similarly, it will be understood that when creating a fusion protein between one polypeptide (here an FST moiety) and an antibody moiety (here preferably a Fab, Fab′ or F(ab′)2), there are two options for fusing any moieties to each other: either the polypeptide (here FST) is fused to the heavy chain of the antibody moiety or to the light chain of the antibody moiety.

Accordingly, in a preferred embodiment of the fusion protein of the invention, the antibody moiety is connected to the C-terminal portion of the follistatin moiety. If a linker is present, the antibody moiety is preferably connected (or conjugated), via the linker, to the C-terminal portion of the follistatin moiety (in other words, the antibody moiety is connected (or conjugated) to the C-terminal portion of the follistatin moiety, and there is a linker between the two moieties). In one example, the fusion protein will comprise (from N-term to C-term) the Follistatin moiety, a linker linked to the C-term of the follistatin moiety and then the heavy chain of the antibody moiety linked to the free-terminus of the linker (typically the C-terminus of the linker).

In some specific (but not limiting) examples, the fusion protein according to the invention comprises or consists of:
(a) i. an FST315 polypeptide defined by SEQ ID NO: 1, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315 polypeptide; and
iii. a Fab light chain defined by SEQ ID NO: 6, or a sequence having at least 95%, at least 96%, at least 97%, at least 98% or at least 99% sequence identity thereto;
(b) i. an FST288 polypeptide defined by SEQ ID NO: 2, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288 polypeptide; and
iii. a Fab light chain defined by SEQ ID NO: 6, or a sequence having at least 95%, at least 96%, at least 97%, at least 98% or at least 99% sequence identity thereto;
(c) i. an FST315HBM polypeptide defined by SEQ ID NO: 3, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315HBM polypeptide; and
iii. a Fab light chain defined by SEQ ID NO: 6, or a sequence having at least 95%, at least 96%, at least 97%, at least 98% or at least 99% sequence identity thereto;
(d) i. an FST288HBM polypeptide defined by SEQ ID NO: 4, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288HBM polypeptide; and
iii. a Fab light chain defined by SEQ ID NO: 6, or a sequence having at least 95%, at least 96%, at least 97%, at least 98% or at least 99% sequence identity thereto;
(e) i. an FST315 polypeptide defined by SEQ ID NO: 1, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(f) i. an FST288 polypeptide defined by SEQ ID NO: 2, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(g) i. an FST315 polypeptide variant defined by SEQ ID NO: 3 (FST315HBM) or SEQ ID NO: 22, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(h) i. an FST288 polypeptide variant defined by SEQ ID NO: 4 (FST288HBM) or SEQ ID NO:25, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(i) SEQ ID NO: 8, 9, 10, 11, 24, 25, 26, 27, 32, 33, 34 or 35 or a sequence having at least 95%, at least 96%, at least 97%, at least 98% or at least 99% sequence identity thereto and a Fab light chain defined by SEQ ID NO: 6, or a sequence having at least 95%, at least 96%, at least 97%, at least 98% or at least 99% sequence identity thereto;
(j) SEQ ID NO: 8, 9, 10, 11, 24, 25, 26, 27, 32, 33, 34 or 35 and a Fab light chain defined by SEQ ID NO: 6; or
(k) a functional variant or fragment of any one of (a) to (i).

Alternatively, should one prefer to use a N-terminus fusion, the invention provides i. a follistatin moiety defined by any one of SEQ ID NOs: 1 to 4, or a sequence having at least 95%, at least 96%, at least 97%, at least 98% or at least 99% sequence identity thereto; ii. a Fab heavy chain defined by SEQ ID NO: 5, or a sequence having at least 95%, at least 96%, at least 97%, at least 98% or at least 99% sequence identity thereto, conjugated to the N-terminus of the FST315 polypeptide; and iii. a Fab light chain defined by SEQ ID NO: 6, or a sequence having at least 95%, at least 96%, at least 97%, at least 98% or at least 99% sequence identity thereto, and optionally a linker between the follistatin moiety and the Fab heavy chain. In some specific examples, the fusion protein can be defined by SEQ ID NO: 28, 29, 30 or 31, or a sequence having at least 95%, at least 96%, at least 97%, at least 98% or at least 99% sequence identity thereto, together with a Fab light chain defined by SEQ ID NO: 6 or a sequence having at least 95%, at least 96%, at least 97%, at least 98% or at least 99% sequence identity thereto.

It was shown that the fusion proteins according to the invention as a whole have an expression level that is at least 1.5 times greater, at least 2 times greater, at least 3 times greater, at least 4 times greater or more compared to the expression level of a wild type FST or of an FST-Fc fusion protein. It was also shown that they result in an overall yield of monomeric protein that is at least 1.5 times, at least 2 times, at least 3 times, at least 4 times, at least 5 times, or at least 6 times greater than the overall yield of monomeric protein of an FST-Fc fusion.

When making such comparison it is preferred to compare like with like, e.g. an FST315-Fab fusion protein should be compared with an FST315-Fc fusion protein; and/or an FST288-Fab fusion protein should be compared with an FST288-Fc fusion protein.

In addition, as highlighted in the examples, they were present in the serum for a longer time period compared to a wild-type FST (6 days or more for the fusion proteins according to the invention compared to 1 day for the wild-type FST).

In a further aspect, the invention provides an isolated polynucleotide encoding the fusion protein according to the invention as a whole, or a functional variant or fragment thereof. The isolated polynucleotide according to the present invention may comprise synthetic DNA, for instance produced by chemical processing, cDNA, genomic DNA or any combination thereof.

Accordingly, herein provided are isolated polynucleotides encoding the fusion proteins according to the invention as a whole, wherein the fusion proteins comprise a follistatin (FST) moiety, an antibody moiety, and optionally a linker between the follistatin moiety and the antibody moiety. The skilled person would understand that said polynucleotide sequence(s) will further comprise a nucleic acid sequence encoding a N-terminal secretion signal sequence. Said sequence will be chosen in particular depending on the host cell that will express the fusion protein.

Standard techniques of molecular biology may be used to prepare DNA sequences coding for the fusion proteins according to the present invention. Desired DNA sequences may be synthesized completely or in part using oligonucleotide synthesis techniques. Site-directed mutagenesis and polymerase chain reaction (PCR) techniques may be used as appropriate. it will be understood that at least two isolated polynucleotides will be needed to encode the fusion proteins according to the inventions. Indeed at least one isolated polynucleotide will encode the FST moiety, the antibody moiety that is fused to the FST moiety and the optional linker in between and another isolated polynucleotide will encode the remaining antibody moiety completing the one fused to the FST moiety. As a non limiting example, one polynucleotide will encode the FST moiety, a linker and the heavy chain of an anti-HSA-Fab moiety and one polynucleotide will encode the light chain of the anti-HSA-Fab moiety.

In some specific (but not limiting) examples, the isolated polynucleotides encode a fusion protein according to the invention comprising or consisting of:
(a) i. an FST315 polypeptide defined by SEQ ID NO: 1, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315 polypeptide; and
iii. a Fab light chain defined by SEQ ID NO: 6, or a sequence having at least 95%, at least 96%, at least 97%, at least 98% or at least 99% sequence identity thereto;
(b) i. an FST288 polypeptide defined by SEQ ID NO: 2, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288 polypeptide; and
iii. a Fab light chain defined by SEQ ID NO: 6, or a sequence having at least 95%, at least 96%, at least 97%, at least 98% or at least 99% sequence identity thereto;
(c) i. an FST315HBM polypeptide defined by SEQ ID NO: 3, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315HBM polypeptide; and
iii. a Fab light chain defined by SEQ ID NO: 6, or a sequence having at least 95%, at least 96%, at least 97%, at least 98% or at least 99% sequence identity thereto;
(d) i. an FST288HBM polypeptide defined by SEQ ID NO: 4, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288HBM polypeptide; and
iii. a Fab light chain defined by SEQ ID NO: 6, or a sequence having at least 95%, at least 96%, at least 97%, at least 98% or at least 99% sequence identity thereto;
(e) i. an FST315 polypeptide defined by SEQ ID NO: 1, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(f) i. an FST288 polypeptide defined by SEQ ID NO: 2, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(g) i. an FST315 polypeptide variant defined by SEQ ID NO: 3 (FST315HBM) or SEQ ID NO: 22, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(h) i. an FST288 polypeptide variant defined by SEQ ID NO: 4 (FST288HBM) or SEQ ID NO:25, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(i) SEQ ID NO: 8, 9, 10, 11, 24, 25, 26, 27, 32, 33, 34 or 35 or a sequence having at least 95%, at least 96%, at least 97%, at least 98% or at least 99% sequence identity thereto and a Fab light chain defined by SEQ ID NO: 6, or a sequence having at least 95%, at least 96%, at least 97%, at least 98% or at least 99% sequence identity thereto;
(j) SEQ ID NO: 8, 9, 10, 11, 24, 25, 26, 27, 32, 33, 34 or 35 and a Fab light chain defined by SEQ ID NO: 6; or
(k) a functional variant or fragment of any one of (a) to (i).

Alternatively, should one prefer to use a N-terminus fusion, the invention provides polynucleotide sequences which encode a fusion protein comprising or consisting of i. a follistatin moiety defined by any one of SEQ ID NOs: 1 to 4, or a sequence having at least 95%, at least 96%, at least 97%, at least 98% or at least 99% sequence identity thereto; ii. a Fab heavy chain defined by SEQ ID NO: 5, or a sequence having at least 95%, at least 96%, at least 97%, at least 98% or at least 99% sequence identity thereto, conjugated to the N-terminus of the FST315 polypeptide; and iii. a Fab light chain defined by SEQ ID NO: 6, or a sequence having at least 95%, at least 96%, at least 97%, at least 98% or at least 99% sequence identity thereto, and optionally a linker between the follistatin moiety and the Fab heavy chain. In some specific examples, the invention provides polynucleotide sequences which encode a fusion protein defined by SEQ ID NO: 28, 29, 30 or 31, or a sequence having at least 95%, at least 96%, at least 97%, at least 98% or at least 99% sequence identity thereto, together with a Fab light chain defined by SEQ ID NO: 6 or a sequence having at least 95%, at least 96%, at least 97%, at least 98% or at least 99% sequence identity thereto.

In some specific (but not limiting) examples, the isolated polynucleotides comprise or consist of: (i) SEQ ID NO: 36, 37, 38, 39, 57 or 58 encoding the follistatin moiety or a sequence having at least 95%, at least 96%, at least 97%, at least 98% or at least 99% sequence identity thereto; (ii) SEQ ID NO: 48, 49 and 50 encoding the CDRs of the heavy chain of the antibody moiety, or a sequence having at least 95%, at least 96%, at least 97%, at least 98% or at least 99% sequence identity thereto; (iii) SEQ ID NO: 51, 52 and 53 encoding the CDRs of the light chain of the antibody moiety and (iv) SEQ ID NO: 42, 54, 55 or 56 encoding the linker should a linker be present.

In further specific (but not limiting) examples, the isolated polynucleotides comprise or consist of: (i) SEQ ID NO: 36, 37, 38, 39, 57 or 58 encoding the follistatin moiety or a sequence having at least 95%, at least 96%, at least 97%, at least 98% or at least 99% sequence identity thereto; (ii) SEQ ID NO: 40 encoding the heavy chain of the antibody moiety, or a sequence having at least 95%, at least 96%, at least 97%, at least 98% or at least 99% sequence identity thereto; (iii) SEQ ID NO: 41 encoding the light chain of the antibody moiety, or a sequence having at least 95%, at least 96%, at least 97%, at least 98% or at least 99% sequence identity thereto; and (iv) SEQ ID NO: 42, 54, 55 or 56 encoding the linker should a linker be present.

In yet other specific (but not limiting) examples, the isolated polynucleotides comprise or consist of: (i) SEQ ID NO: 43, 44, 45, 46, 59, 60, 61, 62, 63, 64, 65 or 66 encoding the Fab heavy chain moiety that is fused to the FST moiety and the optional linker in between or a sequence having at least 95%, at least 96%, at least 97%, at least 98% or at least 99% sequence identity thereto; and (i) SEQ ID NO: 41 encoding the Fab light chain.

In a related aspect, the invention provides cloning or expression vector(s) comprising the polynucleotides encoding the fusion protein according to the invention as a whole. It will be understood that the skilled person as the choice between a double-gene vector (comprising two expressing cassettes, one encoding the FST moiety, the antibody moiety that is fused to the FST moiety and the optional linker in between and another one encoding the remaining antibody moiety) or two different vectors (one encoding the FST moiety, the antibody moiety that is fused to the FST moiety and the optional linker in between and another one encoding the remaining antibody moiety).

General methods by which the vectors may be constructed, transfection methods and culture methods are well known to those skilled in the art. In this respect, reference is made for instance to “Current Protocols in Molecular Biology”, 1999, F. M. Ausubel (ed), Wiley Interscience, New York and the Maniatis Manual produced by Cold Spring Harbor Publishing.

In a related aspect, the invention provides a host cell comprising the polynucleotide sequences encoding the fusion proteins of the invention, or cloning or expression vector(s) comprising one or more polynucleotides encoding the fusion proteins of the invention.

Any suitable host cell/vector system may be used for expression of the polynucleotide sequences encoding the fusion protein of the present invention. Bacterial, for example E. coli, and other microbial systems may be used or eukaryotic, for example mammalian, host cell expression systems may also be used. Suitable mammalian host cells include CHO, myeloma or hybridoma cells. In one embodiment, a host cell comprises (e.g., has been transformed with): (1) a vector comprising two expressing cassettes, one encoding the FST moiety, the antibody moiety that is fused to the FST moiety and the optional linker in between and another one encoding the remaining antibody moiety, or (2) a first vector comprising a nucleic acid that encodes an amino acid sequence comprising or consisting of the FST moiety, the antibody moiety that is fused to the FST moiety and the optional linker in between and a second comprising a nucleic acid that encodes an amino acid sequence comprising or consisting of the remaining antibody moiety.

Suitable host cells for cloning or expression of fusion protein-encoding vectors include prokaryotic or eukaryotic cells described herein. For example, antibodies may be produced in bacteria, in particular when glycosylation and Fc effector function are not needed. For expression of antibody fragments and polypeptides in bacteria, see Charlton, Methods in Molecular Biology, Vol. 248 (B. K. C. Lo, ed., Humana Press, Totowa, NJ, 2003, pp. 245-254, describing expression of antibody fragments in E. coli.). In addition to prokaryotes, eukaryotic microbes such as filamentous fungi or yeast may be suitable cloning or expression hosts for fusion protein-encoding vectors, including fungi and yeast strains whose glycosylation pathways have been “humanized,” resulting in the production of an antibody with a partially or fully human glycosylation pattern (See Gerngross et al, 2004; Li et al., 2006). Alternatively, suitable types of mammalian cells such as Chinese Hamster Ovary (CHO cells) can be used in the present invention, including CHO-S, CHO-K1 cells, dhfr-CHO cells, such as CHO-DG44 cells and CHO-DXB11 cells which can be used with a DHFR selectable marker, or yet CHO-K1 cells or CHOK1-SV cells which can be used with a glutamine synthetase selectable marker. Other cell types of use in expressing antibodies include lymphocytic cell lines, e.g., NSO myeloma cells and SP2 cells, COS cells. The host cell may be stably transformed or transfected with the isolated polynucleotide sequences or the expression vectors according to the present invention.

In a related aspect, the invention provides a process for the production of a fusion protein according to the invention, comprising culturing a host cell of the invention under suitable conditions for producing the fusion protein. The processes according to the invention can further comprise the step of recovering the cell culture fluid (CCF) comprising the fusion protein (harvest step), in other words the step of harvesting the fusion protein. Subsequently to the harvest, the fusion protein may be purified, e.g. using Protein A chromatography and other chromatographic/filtration steps.

The processes further optionally comprise a step of formulating the purified fusion protein, e.g. into a formulation with a protein concentration, such as a concentration of 10 mg/ml or more, e.g. 50 mg/ml or more. Without any limitation, the formulation can be a liquid formulation, lyophilised formulation or a spray-dried formulation. For all these steps, standard processes can be used.

In a further aspect, the invention provides a process for the purification of a fusion protein according to the invention, comprising:
i. Loading a clarified cell culture fluid comprising the fusion proteins according to the invention onto a Protein A chromatographic column, previously equilibrated with a buffer, so that to bind the fusion proteins to the column
ii. Washing the chromatographic column with a wash buffer that is the same as the equilibration buffer of step i. so that to eliminate the impurities,
iii. Eluting the fusion proteins bound to the column with an elution buffer under alkali conditions,
iv. Further eluting any remaining bound fusion proteins with an acidic elution buffer,
v. Neutralising the eluates from steps iii. and iv. so that to obtain neutralised samples
vi. Submitting said neutralised samples to additional purification steps in order to obtain the purified fusion protein.

In a non-limiting example, the equilibration/wash buffer of steps i. and ii. is a sodium acetate buffer at a concentration of from or from about 30 to or to about 70 mM, such as about 30, 35, 40, 45, 50, 55, 56, 57, 58, 59, 60, 61, 62, 63, 64, 65 or 70 mM, and with a pH between about 5.5 to about 6.5, such as a pH of or of about 5.5, 5.6, 5.7, 5.8, 5.9, 6.0, 6.1, 6.2, 6.3, 6.4, or 6.5. In another non-limiting example, the elution buffer of step iii. is a glycine-based buffer, such as glycine/NaOH buffer, at a concentration of from or from about 30 to or to about 70 mM, such as about 30, 35, 40, 45, 46, 47, 48, 49, 50, 51, 52, 53, 54, 55, 60, 65 or 70 mM, and with a pH between about 8.0 to about 9.0, such as a pH of or of about 8.0, 8.1, 8.2, 8.3, 8.4, 8.5, 8.6, 8.7, 8.8, 8.9, 9.0. In yet another non-limiting example, the acidic elution buffer of step iv. is a citrate buffer at a concentration of from or from about 50 to or to about 200 mM, such as about 50, 60, 70, 80, 90, 100, 110, 120, 130, 140, 150, 160, 170, 180, 190 or 200 mM, and with a pH between about 1.5 to about 2.5, such as a pH of or of about 1.5, 1.6, 1.7, 1.8, 1.9, 2.0, 2.1, 2.2, 2.3, 2.4, or 2.5. In a further non-limiting example, neutralisation of step v. is performed at a pH between 7.0 and 9.0, such as 7.0, 7.2, 7.4, 7.6, 7.8, 8.0, 8.1, 8.2, 8.3, 8.4, 8.5, 8.6, 8.7, 8.8, 8.9 or 9.0. The neutralisation is typically performed with Tris or Tris/HCl. It was found by the inventors that not only the fusion proteins of the invention bound to protein A but were capable of being eluted under alkali conditions. By contrast, the free Fab remained strongly bound to protein A under alkali conditions. This feature provides several potential advantages with regards to the downstream process. Firstly, by not using acidic elution conditions it is possible to avoid co-elution of free Fab as the Fab remains strongly bound to Protein A. Additionally, the FST-Fab is not exposed to harsh acidic pH for a prolonged period. Finally, the alkali elution is compatible with subsequent chromatography steps, meaning reduced sample manipulation and therefore allowing for potential increases in yield and recovery.

As a specific (but not limiting) example, herein provided is provides a process for the production of a fusion protein comprising a follistatin moiety (e.g. FST315, FST315HBM, FST288 or FST288HBM) which is linked in C-term with the N-terminus of the heavy chain of a Fab (VH-CH1) via an optional SGGGGS linker. The FST-Fab heavy chain is co-expressed with the Fab light chain (LC) and the heavy and light chains are connected by an intermolecular disulphide bond.

In yet a further aspect, the invention provides a pharmaceutical composition comprising the fusion proteins according to the invention as a whole and one or more pharmaceutically acceptable carriers, excipients or diluents. Pharmaceutical compositions are typically prepared by mixing an active ingredient (herein the fusions proteins according to the invention) having the desired degree of purity with one or more optional pharmaceutically acceptable carriers in the form of dried formulations or aqueous solutions.

Any suitable pharmaceutically acceptable carrier, diluent and/or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carriers may include, but are not limited to buffers; antioxidants; preservatives; hydrophilic polymers; amino acids; monosaccharides, disaccharides, and other carbohydrates; chelating agents; salt-forming counter-ions; and/or non-ionic surfactants. Preferably, said pharmaceutical composition is formulated as a solution, more preferably as an optionally buffered solution. Supplementary active compounds can also be incorporated into the pharmaceutical compositions of the invention. In one embodiment, the pharmaceutical composition is a composition suitable for intravenous or subcutaneous administration. These pharmaceutical compositions are exemplary only and do not limit the pharmaceutical compositions suitable for other administration routes. The pharmaceutical compositions described herein can be packaged in single unit dosage or in multidosage forms.

The fusion protein or pharmaceutical composition of the invention may be administered via one or more routes of administration using one or more of a variety of methods known in the art. As will be appreciated by the skilled artisan, the route and/or mode of administration will vary depending upon the desired results. Examples of routes of administration for fusion proteins or pharmaceutical compositions of the invention include intravenous, intramuscular, intradermal, intraocular, intraperitoneal, subcutaneous, spinal or other parenteral routes of administration, for example by injection or infusion. Alternatively, the fusion protein or pharmaceutical composition of the invention can be administered via a non-parenteral route, such as a topical, epidermal or mucosal route of administration. Where the product is for injection or infusion, it may take the form of a suspension, solution or emulsion in an oily or aqueous vehicle and it may contain additional agents, such as suspending, preservative, stabilizing and/or dispersing agents. Alternatively, the fusion protein or pharmaceutical formulation according to the invention may be provided in dry form, for reconstitution before use with an appropriate sterile liquid. Solid forms suitable for solution in, or suspension in, liquid vehicles prior to injection may also be prepared.

Once formulated, the fusion protein or pharmaceutical formulation of the invention can be administered directly to the subject.

In another aspect, herein provided is a fusion protein or a pharmaceutical composition according to the invention for use in therapy. Alternatively, herein provided is a method for treating a subject in need of a therapy, the method comprising administering a therapeutically effective amount of the fusion protein or the pharmaceutical composition of the invention. In a further alternative, the invention provides the use of the fusion protein or the pharmaceutical composition according to the invention in the manufacture of a medicament for use in therapy.

The therapeutically effective amount will vary depending on the protein or active fragment thereof, the disease and its severity and the age, weight, etc., of the subject to be treated.

In the context of the invention as a whole, a “subject” generally refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). More preferably, the subject is a human.

It should be noted that the above-mentioned embodiments illustrate rather than limit the invention, and that those skilled in the art will be able to design many alternative embodiments without departing from the scope of the invention and in particular of the claims.

### Description of the Amino Acid Sequences

| | | SEQ |
|---|---|---|
| Name | Sequence | ID NO |
| FST315-mature | GNCWLRQAKNGRCQVLYKTELSKEECCSTGRLSTSWTEEDVN | 1 |
| form | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATQLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEW | |
| FST288-mature | GNCWLRQAKNGRCQVLYKTELSKEECCSTGRLSTSWTEEDVN | 2 |
| form (i.e. lacking N- | DNTLFKWMIFNGGAPNCIPCKETCENVDCGPGKKCRMNKKNKP | |
| terminal secretion | RCVCAPDCSNITWKGPVCGLDGKTYRNECALLKARCKEQPELE | |
| signal peptide) | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCN | |
| FST315HBM- | GNCWLRQAKNGRCQVLYKTELSKEECCSTGRLSTSWTEEDVN | 3 |
| mature form | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEW | |
| FST288HBM- | GNCWLRQAKNGRCQVLYKTELSKEECCSTGRLSTSWTEEDVN | 4 |
| mature form | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCN | |
| Fab heavy chain | EVQLLESGGGLVQPGGSLRLSCAVSGIDLSNYAINWVRQAPGK | 5 |
| (FabHC) | GLEWIGIIWASGTTFYATWAKGRFTISRDNSKNTVYLQMNSLRA | |
| | EDTAVYYCARTVPGYSTAPYFDLWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSC | |
| Fab light chain | DIQMTQSPSSVSASVGDRVTITCQSSPSVWSNFLSWYQQK | 6 |
| (FabLC) | PGKAPKLLIYEASKLTSGVPSRFSGSGSGTDFTLTISSLQPEDFA | |
| | TYYCGGGYSSISDTTFGGGTKVEIKRTVAAPSVFIFPPSDEQLKS | |
| | GTASVVCLLNNFYPREAKVQWKVDNALQSGNSQESVTEQDSK | |
| | DSTYSLSSTLTLSKADYEKHKVYACEVTHQGLSSPVTKSFNRGE | |
| | C | |
| Exemplary Linker | SGGGGS | 7 |
| FST315HBM-FabHC | GNCWLRQAKNGRCQVLYKTELSKEECCSTGRLSTSWTEEDVN | 8 |
| (with linker | DNTLFKWMIFNGGAPNCIPCKETCENVDCGPGKACRMNAANKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWSGGGGSEVQLLESGGGLVQPGGSLRLSC | |
| | AVSGIDLSNYAINWVRQAPGKGLEWIGIIWASGTTFYATWAKGR | |
| | FTISRDNSKNTVYLQMNSLRAEDTAVYYCARTVPGYSTAPYFDL | |
| | WGQGTLVTVSSASTKGPSVFPLAPSSKSTSGGTAALGCLVKDY | |
| | FPEPVTVSWNSGALTSGVHTFPAVLQSSGLYSLSSVVTVPSSSL | |
| | GTQTYICNVNHKPSNTKVDKKVEPKSC | |
| FST315HBM-FabHC | GNCWLRQAKNGRCQVLYKTELSKEECCSTGRLSTSWTEEDVN | 9 |
| (without linker) | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWEVQLLESGGGLVQPGGSLRLSCAVSGIDL | |
| | SNYAINWVRQAPGKGLEWIGIIWASGTTFYATWAKGRFTISRDN | |
| | SKNTVYLQMNSLRAEDTAVYYCARTVPGYSTAPYFDLWGQGTL | |
| | VTVSSASTKGPSVFPLAPSSKSTSGGTAALGCLVKDYFPEPVTV | |
| | SWNSGALTSGVHTFPAVLQSSGLYSLSSVVTVPSSSLGTQTYIC | |
| | NVNHKPSNTKVDKKVEPKSC | |
| FST288HBM-FabHC | GNCWLRQAKNGRCQVLYKTELSKEECCSTGRLSTSWTEEDVN | 10 |
| (with linker | DNTLFKWMIFNGGAPNCIPCKETCENVDCGPGKACRMNAANKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSGGGGSEVQLL | |
| | ESGGGLVQPGGSLRLSCAVSGIDLSNYAINWVRQAPGKGLEWI | |
| | GIIWASGTTFYATWAKGRFTISRDNSKNTVYLQMNSLRAEDTAV | |
| | YYCARTVPGYSTAPYFDLWGQGTLVTVSSASTKGPSVFPLAPS | |
| | SKSTSGGTAALGCLVKDYFPEPVTVSWNSGALTSGVHTFPAVL | |
| | QSSGLYSLSSVVTVPSSSLGTQTYICNVNHKPSNTKVDKKV | |
| | EPKSC | |
| FST288HBM-FabHC | GNCWLRQAKNGRCQVLYKTELSKEECCSTGRLSTSWTEEDVN | 11 |
| (without linker) | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNEVQLLESGGGLV | |
| | QPGGSLRLSCAVSGIDLSNYAINWVRQAPGKGLEWIGIIWASGT | |
| | TFYATWAKGRFTISRDNSKNTVYLQMNSLRAEDTAVYYCARTV | |
| | PGYSTAPYFDLWGQGTLVTVSSASTKGPSVFPLAPSSKSTSGG | |
| | TAALGCLVKDYFPEPVTVSWNSGALTSGVHTFPAVLQ | |
| | SSGLYSLSSVVTVPSSSLGTQTYICNVNHKPSNTKVDKKV | |
| | EPKSC | |
| N-terminal secretion | MVRARHQPGGLCLLLLLLCQFMEDRSAQA | 12 |
| signal | | |
| sequence/peptide | | |
| Light Chain CDR1 | QSSPSVWSNFLS | 13 |
| Light Chain CDR2 | EASKLTS | 14 |
| Light Chain CDR3 | GGGYSSISDTT | 15 |
| Heavy Chain CDR1 | GIDLSNYAIN | 16 |
| Heavy Chain CDR2 | IIWASGTTFYATWAKG | 17 |
| Heavy Chain CDR3 | TVPGYSTAPYFDL | 18 |
| Alternative | SGGGGSSGGGGS | 19 |
| Exemplary Linker | | |
| Alternative | GGGGS | 20 |
| Exemplary Linker | | |
| Alternative | GGGGSGGGGS | 21 |
| Exemplary Linker | | |
| FST315(variant) | GNCWLRQAKNGRCQVLYKTELSKEECCSTGRLSTSWTEEDVN | 22 |
| | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEW | |
| FST288(variant) | GNCWLRQAKNGRCQVLYKTELSKEECCSTGRLSTSWTEEDVN | 23 |
| | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCN | |
| FST315(variant)- | GNCWLRQAKNGRCQVLYKTELSKEECCSTGRLSTSWTEEDVN | 24 |
| FabHC | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWEVQLLESGGGLVQPGGSLRLSCAVSGIDL | |
| | SNYAINWVRQAPGKGLEWIGIIWASGTTFYATWAKGRFTISRDN | |
| | SKNTVYLQMNSLRAEDTAVYYCARTVPGYSTAPYFDLWGQGTL | |
| | VTVSSASTKGPSVFPLAPSSKSTSGGTAALGCLVKDYFPEPVTV | |
| | SWNSGALTSGVHTFPAVLQSSGLYSLSSVVTVPSSSLGTQTYIC | |
| | NVNHKPSNTKVDKKVEPKSC | |
| FST315(variant)- | GNCWLRQAKNGRCQVLYKTELSKEECCSTGRLSTSWTEEDVN | 25 |
| FabHC (with linker | DNTLFKWMIFNGGAPNCIPCKETCENVDCGPGKXCRMNXXNKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWSGGGGSEVQLLESGGGLVQPGGSLRLSC | |
| | AVSGIDLSNYAINWVRQAPGKGLEWIGIIWASGTTFYATWAKGR | |
| | FTISRDNSKNTVYLQMNSLRAEDTAVYYCARTVPGYSTAPYFDL | |
| | WGQGTLVTVSSASTKGPSVFPLAPSSKSTSGGTAALGCLVKDY | |
| | FPEPVTVSWNSGALTSGVHTFPAVLQSSGLYSLSSVVTVPSSSL | |
| | GTQTYICNVNHKPSNTKVDKKVEPKSC | |
| FST288(variant)- | GNCWLRQAKNGRCQVLYKTELSKEECCSTGRLSTSWTEEDVN | 26 |
| FabHC | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNEVQLLESGGGLV | |
| | QPGGSLRLSCAVSGIDLSNYAINWVRQAPGKGLEWIGIIWASGT | |
| | TFYATWAKGRFTISRDNSKNTVYLQMNSLRAEDTAVYYCARTV | |
| | PGYSTAPYFDLWGQGTLVTVSSASTKGPSVFPLAPSSKSTSGG | |
| | TAALGCLVKDYFPEPVTVSWNSGALTSGVHTFPAVLQSSGLYSL | |
| | SSVVTVPSSSLGTQTYICNVNHKPSNTKVDKKVEPKSC | |
| FST288(variant)- | GNCWLRQAKNGRCQVLYKTELSKEECCSTGRLSTSWTEEDVN | 27 |
| FabHC (with linker | DNTLFKWMIFNGGAPNCIPCKETCENVDCGPGKXCRMNXXNKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSGGGGSEVQLL | |
| | ESGGGLVQPGGSLRLSCAVSGIDLSNYAINWVRQAPGKGLEWI | |
| | GIIWASGTTFYATWAKGRFTISRDNSKNTVYLQMNSLRAEDTAV | |
| | YYCARTVPGYSTAPYFDLWGQGTLVTVSSASTKGPSVFPLAPS | |
| | SKSTSGGTAALGCLVKDYFPEPVTVSWNSGALTSGVHTFPAVL | |
| | QSSGLYSLSSVVTVPSSSLGTQTYICNVNHKPSNTKVDKKVEPK | |
| | SC | |
| FabHC- | EVQLLESGGGLVQPGGSLRLSCAVSGIDLSNYAINWVRQAPGK | 28 |
| FST315(variant) | GLEWIGIIWASGTTFYATWAKGRFTISRDNSKNTVYLQMNSLRA | |
| | EDTAVYYCARTVPGYSTAPYFDLWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCGNCWLRQAKNGRCQVLYKTELSKEECCSTGRLSTS | |
| | WTEEDVNDNTLFKWMIFNGGAPNCIPCKETCENVDCGPGKXC | |
| | RMNXXNKPRCVCAPDCSNITWKGPVCGLDGKTYRNECALLKA | |
| | RCKEQPELEVQYQGRCKKTCRDVFCPGSSTCVVDQTNNAYCV | |
| | TCNRICPEPASSEQYLCGNDGVTYSSACHLRKATCLLGRSIGLA | |
| | YEGKCIKAKSCEDIQCTGGKKCLWDFKVGRGRCSLCDELCPDS | |
| | KSDEPVCASDNATYASECAMKEAACSSGVLLEVKHSGSCNSIS | |
| | EDTEEEEEDEDQDYSFPISSILEW | |
| FabHC- | EVQLLESGGGLVQPGGSLRLSCAVSGIDLSNYAINWVRQAPGK | 29 |
| FST315(variant)- | GLEWIGIIWASGTTFYATWAKGRFTISRDNSKNTVYLQMNSLRA | |
| (with linker | EDTAVYYCARTVPGYSTAPYFDLWGQGTLVTVSSASTKGPSVF | |
| underlined) | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCSGGGGSGNCWLRQAKNGRCQVLYKTELSKEECCST | |
| | GRLSTSWTEEDVNDNTLFKWMIFNGGAPNCIPCKETCENVDCG | |
| | PGKXCRMNXXNKPRCVCAPDCSNITWKGPVCGLDGKTYRNEC | |
| | ALLKARCKEQPELEVQYQGRCKKTCRDVFCPGSSTCVVDQTN | |
| | NAYCVTCNRICPEPASSEQYLCGNDGVTYSSACHLRKATCLLG | |
| | RSIGLAYEGKCIKAKSCEDIQCTGGKKCLWDFKVGRGRCSLCD | |
| | ELCPDSKSDEPVCASDNATYASECAMKEAACSSGVLLEVKHSG | |
| | SCNSISEDTEEEEEDEDQDYSFPISSILEW | |
| FabHC- | EVQLLESGGGLVQPGGSLRLSCAVSGIDLSNYAINWVRQAPGK | 30 |
| FST288(variant) | GLEWIGIIWASGTTFYATWAKGRFTISRDNSKNTVYLQMNSLRA | |
| | EDTAVYYCARTVPGYSTAPYFDLWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCGNCWLRQAKNGRCQVLYKTELSKEECCSTGRLSTS | |
| | WTEEDVNDNTLFKWMIFNGGAPNCIPCKETCENVDCGPGKXC | |
| | RMNXXNKPRCVCAPDCSNITWKGPVCGLDGKTYRNECALLKA | |
| | RCKEQPELEVQYQGRCKKTCRDVFCPGSSTCVVDQTNNAYCV | |
| | TCNRICPEPASSEQYLCGNDGVTYSSACHLRKATCLLGRSIGLA | |
| | YEGKCIKAKSCEDIQCTGGKKCLWDFKVGRGRCSLCDELCPDS | |
| | KSDEPVCASDNATYASECAMKEAACSSGVLLEVKHSGSCN | |
| FabHC- | EVQLLESGGGLVQPGGSLRLSCAVSGIDLSNYAINWVRQAPGK | 31 |
| FST288(variant)- | GLEWIGIIWASGTTFYATWAKGRFTISRDNSKNTVYLQMNSLRA | |
| (with linker | EDTAVYYCARTVPGYSTAPYFDLWGQGTLVTVSSASTKGPSVF | |
| underlined) | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCSGGGGSGNCWLRQAKNGRCQVLYKTELSKEECCST | |
| | GRLSTSWTEEDVNDNTLFKWMIFNGGAPNCIPCKETCENVDCG | |
| | PGKXCRMNXXNKPRCVCAPDCSNITWKGPVCGLDGKTYRNEC | |
| | ALLKARCKEQPELEVQYQGRCKKTCRDVFCPGSSTCVVDQTN | |
| | NAYCVTCNRICPEPASSEQYLCGNDGVTYSSACHLRKATCLLG | |
| | RSIGLAYEGKCIKAKSCEDIQCTGGKKCLWDFKVGRGRCSLCD | |
| | ELCPDSKSDEPVCASDNATYASECAMKEAACSSGVLLEVKHSG | |
| | SCN | |
| FST315-FabHC (with | GNCWLRQAKNGRCQVLYKTELSKEECCSTGRLSTSWTEEDVN | 32 |
| linker underlined) | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWSGGGGSEVQLLESGGGLVQPGGSLRLSC | |
| | AVSGIDLSNYAINWVRQAPGKGLEWIGIIWASGTTFYATWAKGR | |
| | FTISRDNSKNTVYLQMNSLRAEDTAVYYCARTVPGYSTAPYFDL | |
| | WGQGTLVTVSSASTKGPSVFPLAPSSKSTSGGTAALGCLVKDY | |
| | FPEPVTVSWNSGALTSGVHTFPAVLQSSGLYSLSSVVTVPSSSL | |
| | GTQTYICNVNHKPSNTKVDKKVEPKSC | |
| FST315-FabHC | GNCWLRQAKNGRCQVLYKTELSKEECCSTGRLSTSWTEEDVN | 33 |
| (without linker) | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWEVQLLESGGGLVQPGGSLRLSCAVSGIDL | |
| | SNYAINWVRQAPGKGLEWIGIIWASGTTFYATWAKGRFTISRDN | |
| | SKNTVYLQMNSLRAEDTAVYYCARTVPGYSTAPYFDLWGQGTL | |
| | VTVSSASTKGPSVFPLAPSSKSTSGGTAALGCLVKDYFPEPVTV | |
| | SWNSGALTSGVHTFPAVLQSSGLYSLSSVVTVPSSSLGTQTYIC | |
| | NVNHKPSNTKVDKKVEPKSC | |
| FST288-FabHC (with | GNCWLRQAKNGRCQVLYKTELSKEECCSTGRLSTSWTEEDVN | 34 |
| linker underlined) | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSGGGGSEVQLL | |
| | ESGGGLVQPGGSLRLSCAVSGIDLSNYAINWVRQAPGKGLEWI | |
| | GIIWASGTTFYATWAKGRFTISRDNSKNTVYLQMNSLRAEDTAV | |
| | YYCARTVPGYSTAPYFDLWGQGTLVTVSSASTKGPSVFPLAPS | |
| | SKSTSGGTAALGCLVKDYFPEPVTVSWNSGALTSGVHTFPAVL | |
| | QSSGLYSLSSVVTVPSSSLGTQTYICNVNHKPSNTKVDKKVEPK | |
| | SC | |
| FST288-FabHC | GNCWLRQAKNGRCQVLYKTELSKEECCSTGRLSTSWTEEDVN | 35 |
| (without linker) | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNEVQLLESGGGLV | |
| | QPGGSLRLSCAVSGIDLSNYAINWVRQAPGKGLEWIGIIWASGT | |
| | TFYATWAKGRFTISRDNSKNTVYLQMNSLRAEDTAVYYCARTV | |
| | PGYSTAPYFDLWGQGTLVTVSSASTKGPSVFPLAPSSKSTSGG | |
| | TAALGCLVKDYFPEPVTVSWNSGALTSGVHTFPAVLQSSGLYSL | |
| | SSVVTVPSSSLGTQTYICNVNHKPSNTKVDKKVEPKSC | |

* The Xs in SEQ ID Nos 22-31 stand “for any amino acid”

### EXAMPLES

### Materials & Methods

### Production of Follistatin-Fab Fusion Proteins

Cloning strategy: DNA segments corresponding to fusions between follistatin and anti-albumin antibody (designated 645 Fab) heavy or light chain sequences (with or without linker sequences in between) were generated by PCR or gene synthesis and cloned using in-house mammalian expression vectors. The heavy and light chain sequences of the 645 Fab were cloned also separately using in-house mammalian expression vectors. All expression vectors were confirmed by direct sequencing using primers which covered the whole open reading frame.

Cultivating CHO cells: Suspensions of CHOS-XE cells (Cain et al., 2013) were pre-adapted in CD CHO medium (Invitrogen) supplemented with 2 mM Glutamax. Cells were kept in logarithmic growth phase with agitation at 120 RPM on a shaking incubator (Kuhner AG) and cultured at 37° C. in an atmosphere containing 8% CO₂.

Protein expression: The follistatin-Fab proteins were overexpressed by transient transfection of the CHO-XE cell line. Pairs of expression plasmids were co-transfected (e.g. N-fab light chain-FST-C with the heavy chain or FST-C fab heavy chain-C with the light chain). Immediately prior to transfection with DNA, CHO cells were exchanged in to Expi CHO expression medium (Gibco) by briefly centrifuging the cells at 1500×g and resuspending the pellet. Cells were then transfected using ExpiFectamine (Gibco), following manufacturer's instructions. The cultures were grown at 37° C. for the first 24 hours and then at 32° C. for the remainder of the expression cycle with shaking at 190 RPM in an atmosphere containing 8% CO₂. Supernatants were typically harvested 9-14 days post-transfection by centrifugation at 4000×g with subsequent filtration using 0.22 μm membranes. Final protein expression levels were determined by Protein G-HPLC and by SDS PAGE.

Protein purification: Transiently expressed protein content was captured using a Mab Select column (GE Healthcare) run under standard conditions. In short, the resin was washed with 10 column volumes of phosphate buffer saline (PBS, pH 7.4), and bound proteins were eluted with 5 column volumes of 0.1 M sodium citrate pH 3.1 (except if mentioned otherwise in the following examples). The eluate was neutralised with TRIS-HCl pH 8.5 and filter-sterilised through 0.22 μm membrae-exclusion chromatography (HiLoad 26/60 Superdex 75 column, GE Healthcare) run under standard conditions (here, columns were preloaded with PBS pH 7.4 as the running buffer). Sample quality was assessed using absorbance at 280 nm, BEH2000 analytical UPLC and SDS PAGE (under reducing and non-reducing conditions).

For protein G purifications, clarified supernatants were loaded onto a protein G HP (GE Healthcare) column equilibrated in PBS pH 7.4 and subsequently washed with the same buffer. Bound material was eluted with 0.1 M Glycine-HCl pH 3.0. Acidic eluates were neutralised with 2M Tris/HCl pH8.5. Purified material was quantified by absorbance at 280 nm.

Size Exclusion Chromatography (SEC): Samples were injected onto a BEH200, 200 Å, 1.7 μm, 4.6 mm ID×300 mm column (Aquity) and developed with an isocratic gradient of 0.2M phosphate pH7 at 0.35 mL/min, with detection by absorbance at 280 nm and multi-channel fluorescence (FLR) detector (Waters).

SDS-PAGE: For analysis by sodium dodecyl sulfate-polyacrylamide gel electrophoresis (SDS-PAGE) samples were prepared by adding 4× Novex NuPAGE LDS sample buffer (Life Technologies) and 100 mM N-ethylmaleimide (Sigma-Aldrich) to purified protein and were heated to 100° C. for 3 min. The samples were loaded onto a 10 well Novex 4-20% Tris-glycine 1.0 mm SDS-polyacrylamide gel (Life Technologies) and separated at a constant voltage of 225V for 40 min in Tris-glycine SDS running buffer (Life Technologies). Novex Mark12 wide-range protein standards (Life Technologies) were used as standards. The gel was stained with Coomassie Quick Stain (Generon) and destained in distilled water.

Pharmacokinetic measurements: FST proteins were dosed to C57BL/6 mice via intravenous administrations at 10 mg/kg (2 mL/kg IV, 5 mL/kg SC). Blood samples were taken daily over 7 days. Serum samples were generated and analysed using a ligand-binding assay detecting follistatin (including FST288 and FST315). Pharmacokinetic parameters were calculated based on the individual data using Phoenix v8.3.

Functional activity reporter gene cell assays: To measure the efficacy of follistatin proteins in blocking the stimuli (Activin A/B, GDF8/11)-evoked activation of the SMAD2/3 signalling pathway, HEK-Blue™ TGFβ reporter cells (Invivogen) were used. Briefly, follistatin proteins and their matching controls were prediluted in the culture medium based on concentration and predicted activity such that the resultant inhibition curve had complete top and bottom. This was followed by 10-point serial dilution at 1 in 3 in the culture medium before aliquoting four replicates of each dilution at 20 μL/well in a 384-cell culture assay plate. 10 μl of each stimulus was added to wells representing the follistatin serial dilution as well as in wells without follistatin representing highest response to stimuli. The wells with matching volume of medium were represented as untreated. The assay plate was incubated for an hour at 37° C. before HEK-Blue™ TGFβ cells (10,000 cells/20 μL) were added per well and the assay plate was incubated at 37° C. for further 17 hours. 45 μL/well of QUANTI-Blue™ solution was added to a fresh 384-assay plate designated as the destination plate, before using automated liquid handlers for transferring 5 μL of cell supernatant from the assay plate to the destination plate. The destination plate was briefly kept on the shaker followed by incubation for an hour at 37° C. The absorbance values from each well were measured at 630 nm on a plate reader and follistatin-mediated dose-dependent percentage inhibition of SMAD2/3 activity was calculated alongside the Z-factor.

### Biacore Binding Data

The binding kinetics of the fusion proteins to various targets (see below) were determined by surface plasmon resonance using a Biacore T200 (Cytiva). For each type of assay, kinetic parameters were determined using a 1:1 binding model using Biacore T200 Evaluation software (version 3.0).

For the assessment of binding to Activin A and Activin B (R&D Systems), a goat anti-human F(ab′)₂ fragment specific antibody (Jackson ImmunoResearch) was first immobilised on a CM5 Sensor Chip via amine coupling chemistry to a level of approximately 5000RU. Using a standard multi-cycle kinetic approach, each analysis cycle consisted of capture of FST-Fab to the anti-F(ab′)₂ surface followed by injection of analyte and finally surface regeneration using 60s injections of 50 mM HCl, and 5 mM NaOH. Analytes were injected (300s at 25° C. at a flow rate of 30 μl/min) using a 3-fold serial dilution in HBS-EP+ running buffer (Cytiva) at concentrations of 30, 10, 3.3, 1.1, 0.367 and 0.122 nM, after which dissociation was monitored for 900s. The binding response of a parallel blank surface was subtracted, and buffer blank injections were included to subtract instrument noise and drift.

For the assessment of binding to GDF8 and GDF11 (R&D Systems), each one of GDF8 and GDF11 was immobilised by amine coupling chemistry to the surface of a CM5 sensorchip to achieve an immobilisation level of approximately 250RU. For both GDF8 and GDF11, analysis was conducted using a single-cycle kinetics approach with sequential 180s injections of FST-Fab at increasing concentrations (0.8, 4, 20, 100 and 500 nM) in HBS-EP+ running buffer (Cytiva) at 25° C. at a flow rate of 30 μl/min, followed by monitoring the dissociation for 1800s. The binding response of a parallel blank surface was subtracted, and a series of buffer blank injections were conducted to subtract instrument noise and drift.

For the assessment of binding to albumin: A goat anti-human F(ab′)2 fragment specific antibody (Jackson ImmunoResearch) was first immobilised on a CM5 Sensor Chip via amine coupling chemistry to a level of approximately 5000RU. Using a standard multi-cycle kinetic approach, each analysis cycle consisted of capture of the fusion protein to be tested to the anti-F(ab′)2 surface followed by injection of albumin and finally surface regeneration using 60s injections of 50 mM HCl, and 5 mM NaOH. Analytes were injected (300s at 25° C. at a flow rate of 30 μl/min) using a 2-fold serial dilution in HBS-EP+ running buffer (Cytiva) at concentrations of 100, 50, 25, 12.5, 6.3 and 3.1 nM, after which dissociation was monitored for 900s. The binding response of a parallel blank surface was subtracted and buffer blank injections were included to subtract instrument noise and drift.

### Example 1. FST-Fab-Leads to Superior Expression Levels and Monomer Yield Compared to Other Fusion Partners

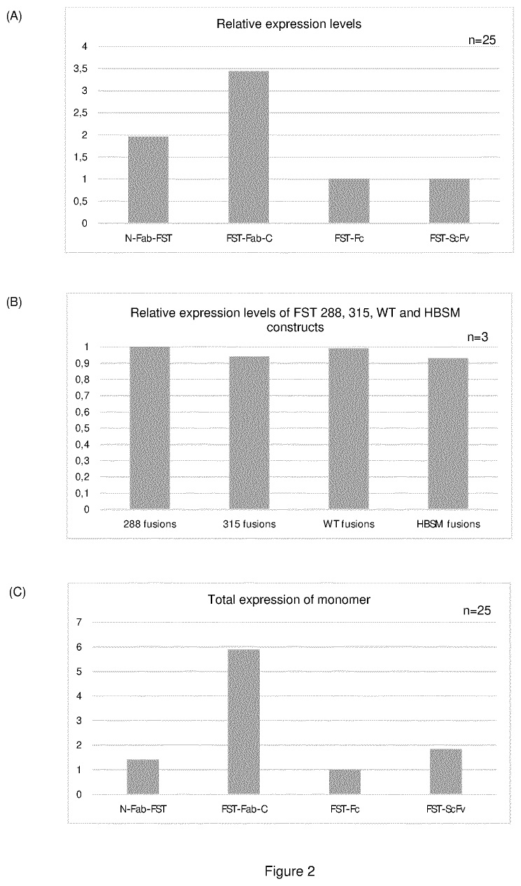

Comparing the relative expression levels of follistatin moiety fused with various fusion partners and in different orientations reveals the FST-Fab constructs fused in C-terminal of the FST moiety to be optimal (FIG. 2A). Having an Fc or a ScFv fused to the C-terminus of FST is significantly inferior to using a Fab at this position, both the former giving a 3.5-fold reduction in expressed product (FIG. 2A). Fusing a Fab moiety at the N-terminus of FST moiety leads to an about 45% drop in the amount of expressed product, although being better than fusion proteins comprising FST fused to an Fc domain. An assessment comparing the effect on expression using FST288- or FST315 fused to a Fab at the C-terminus of follistatin using wild-type of HBM sequences revealed only marginal differences (FIG. 2B). The FST315 version had expression 6% lower than the FST288 fusion. A comparison of fusions which contained the HBSM (HBM) revealed a modest 7% lowering in expression levels compared to wild-type.

A head-to-head examination of the various FST-fusions revealed Fab moieties fused to the C-terminus of the FST moiety to give the highest level of final monomer yield compared to the other fusions (FIG. 2C). The FST-Fc fusion gave the lowest yield of monomer amongst the set, being almost 6-fold worse than the FST-Fab fusion. The FST-ScFv and FST fused at the N-terminus of a Fab were approximately 3 and 4 fold worse, respectively.

### Example 2—FST315(HBM)-Fab Displays Extended Pharmacokinetic Properties Compared to FST315WT in Mice Studies

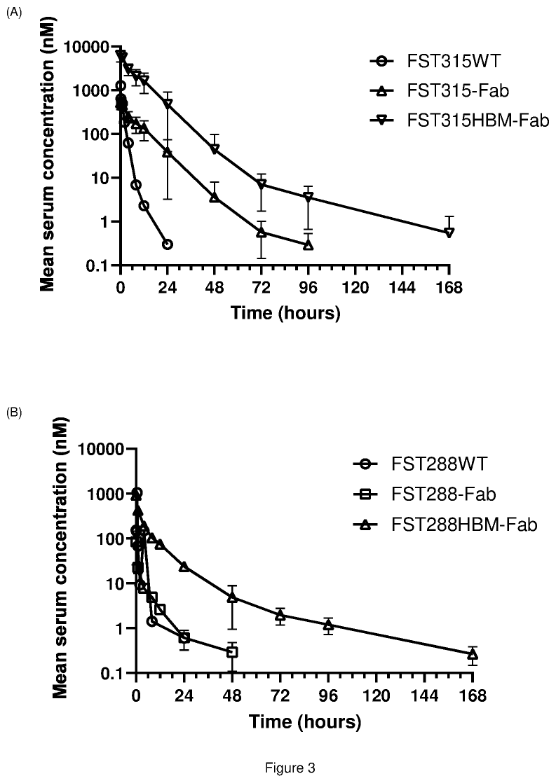

FST315WT, FST288WT and FST288-Fab administered intravenous (IV) at 10 mg/kg into mice were cleared very rapidly, with a mean residence time (MRT) of 1.6 hours, 2.3 hours and 5.2 hours, respectively. By comparison, FST315-Fab, FST315(HBM)-Fab and FST288HBM-Fab administered IV at 10 mg/kg into mice displayed extended kinetics, with an MRT of 9.3 hours, 13.1 hours and 11 hours, respectively, (FIGS. 3A and B). All the pharmacokinetic parameters are summarized in Table 1.

Conclusion: Example 2 shows that it was possible to greatly extend the kinetics and half-life of a FST-containing protein thanks to the fusion between a FST moiety and a Fab moiety. A significant contribution to extended kinetics is also contributed by the mutation of the heparin binding site in the form of the HBM versions of the follistatin moieties.

### Example 3—FST315-Fab, FST315HBM-Fab, FST288-Fab and FST288HBM-Fab Display High Affinity Binding Towards its Ligands and Albumin

Follistatin has four high affinity ligands—Activin A, Activin B, GDF8 (myostatin) and GDF11. The binding of FST315-Fab, FST315HBM-Fab, FST288-Fab and FST288HBM-Fab to these ligands has been confirmed using surface plasma resonance (SPR) binding methods and confirmed the Kd binding affinity is within the expected range as summarized in Table 2 (in view of literature, see Sidis et al., 2006). The affinity Kd values obtained for all the follistatin fusion proteins for their specific ligands agree well with literature values described for wild-type, unconjugated FST288WT and FST315WT binding to activin A of 23.6 pM and 28.7 pM, respectively (Sidis et al., 2006). The human albumin-binding properties of the Fab domain component of FST315(HBM)-Fab was also confirmed by SPR and the value of 2602 pM was in line with expectations for an active albumin binder. The Kd value for FST315(HBM)-Fab albumin binding also agrees closely with previously published values for Fab alone human albumin binding (which is quoted in Adams et al., 2016, as 2-5 nM; referring to different formats of 645gL4gH5 Fab).

Conclusion: Example 2 underlines that the expected biological activities of the FST-Fab moieties were maintained, i.e. the fusion between the two moieties does not impact the binding activities of either the follistatin moiety to their respective biological ligands or the Fab moiety to albumin.

### Example 4—FST315(HBM)-Fab and FST288(HBM)-Fab Display Functional Inhibition Towards its Ligands in a Reporter Gene Cell Assay

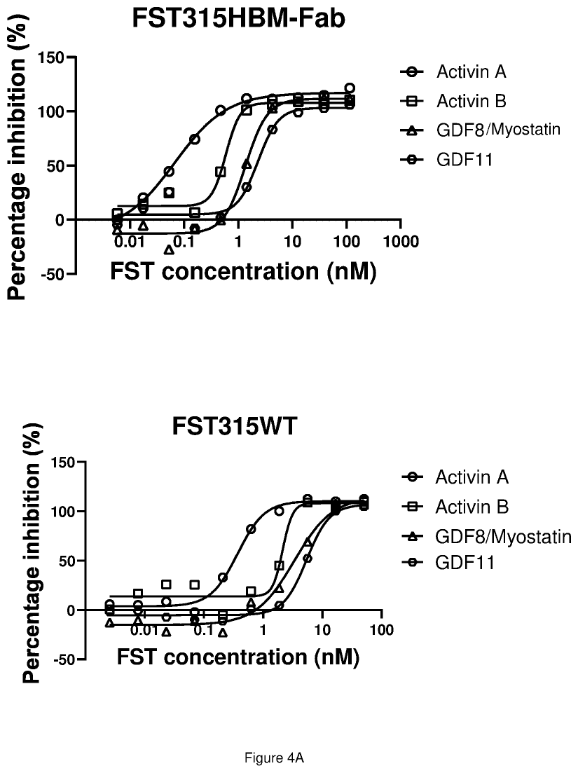

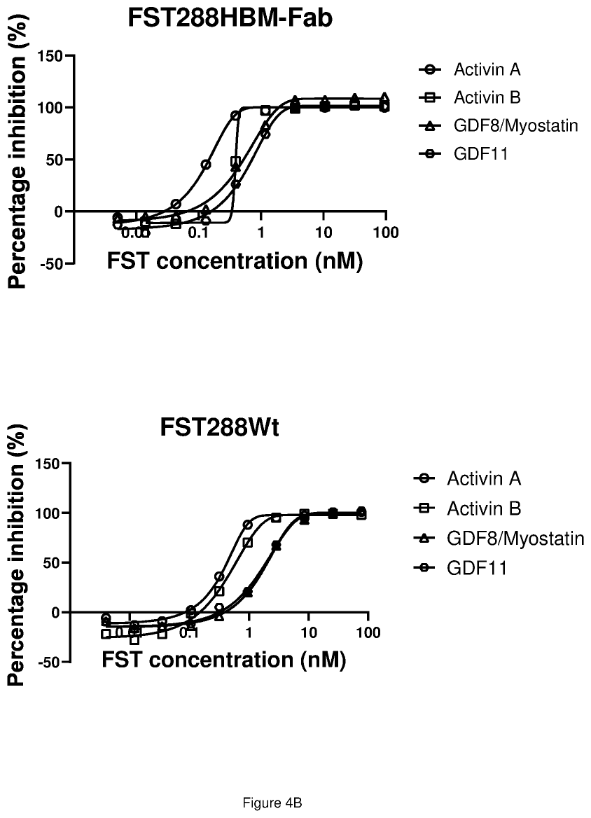

The FST315(HBM)-Fab was next tested for its ability to inhibit the functional signalling of the four ligands using the Smad2/3 reporter gene cell assay, performed in the HEK-Blue™-TGFβ commercial cell line. All four ligands were used at approximately their EC50 concentration for inducing a stimulation of reporter gene activation, and then FST315(HBM)-Fab and FST315WT were titrated over a large concentration range to generate the dose-response curves represented in FIG. 4A. The geomean IC50 data for all four ligands are summarized in Table 3. It was observed that the FST315(HBM)-Fab format was consistently 3-fold more potent than the parent FST315WT when response was induced with the ligands Activin A and Activin B, and 2-fold more potent when the ligands GDF8 and GDF11 were employed. Similarly, FST288(HBM)-Fab and FST288WT were titrated over a large concentration range to generate dose-response curves against all four ligands used at their approximate EC50 concentrations; representative data is presented in FIG. 4B and geomean IC50 data for all four ligands is summarized in Table 3. Unlike the data for FST315 formats, the FST288(HBM)-Fab molecule format demonstrated very similar efficacy to the FST288WT parent molecule across all four ligands.

Conclusion: Not only the example shows that FST315(HBM)-Fab was not inferior to the FST315WT protein in its ability to inhibit ligand induced signalling through the Smad2/3 reporter pathway, but FST315(HBM)-Fab presented an improved potency compared to FST315WT, highlighting its relevance in a therapeutical setting. This contrasts with FST288(HBM)-Fab potency, which was very comparable with the FST288WT molecule.

### Example 5—FST-Fab Fusions with a Human VH3 Domain Allows Recovery Via Protein a Chromatography

FST315HBM-Fab fusions (FST-Fab1) were prepared where the FST is fused to an anti-albumin F(ab′) which contains a human VH3 domain (fusion protein of SEQ ID NO. 8) which enables protein A chromatography.

Two alternative FST-F(ab′) constructs were prepared which do not comprise a human VH3 domain (FST-Fab2 and FST-Fab3). All constructs were expressed and purified according to the methods set out above. As shown in Table 4, only FST-Fab1 which contained a human VH3 domain was recovered following protein A chromatography. All three fusion proteins were able to be recovered following protein G chromatography.

Typically, elution of proteins bound to Protein A affinity capture resins is carried out under acidic conditions (see the section material & methods above). However it was a finding from the inventors that FST-Fab1 of the invention elutes efficiently from protein A under mildly alkal conditions, whereas the Fab fraction remains strongly bound.

FST-Fab 1 clarified supernatants were loaded onto a MabSelect (GE Healthcare) column equilibrated in 50 mM Sodium Acetate pH5.8 and subsequently washed with the same buffer. Bound material was eluted under acidic (0.1 M Glycine-HCl pH2.6) or alkali (50 mM Glycine-NaOH pH8.6) conditions. This was then followed with a further acidic strip (0.1M Citrate pH2.0). Acidic eluates & strip pools were neutralised with 2M Tris/HCl pH8.5. These elution and strip samples were then analysed by SDS-PAGE and analytical size exclusion.

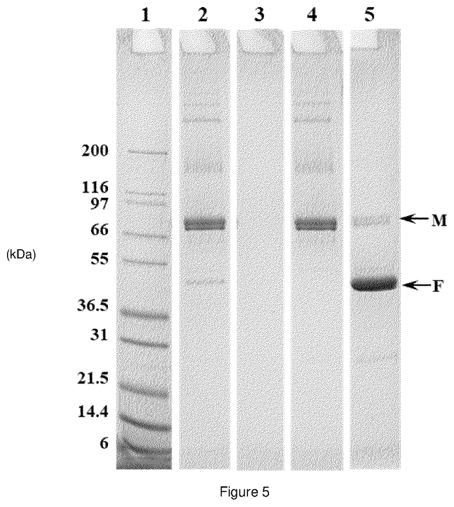

The results are shown in FIG. 5. Analysis by SDS-PAGE shows that upon elution under acidic conditions (lane 2) there is a band at ˜50 kDa indicating that any Fab produced during expression is eluted under these conditions. Lane 3 containing the subsequent strip contains no protein as it has all been removed by the acidic elution. Alternatively, under slightly alkali conditions (lane 4) there is no band present for the Fab. The Fab is not eluted from the column until the acidic strip (lane 5) where there is a band at ˜50 kDa as seen in the acidic elution.

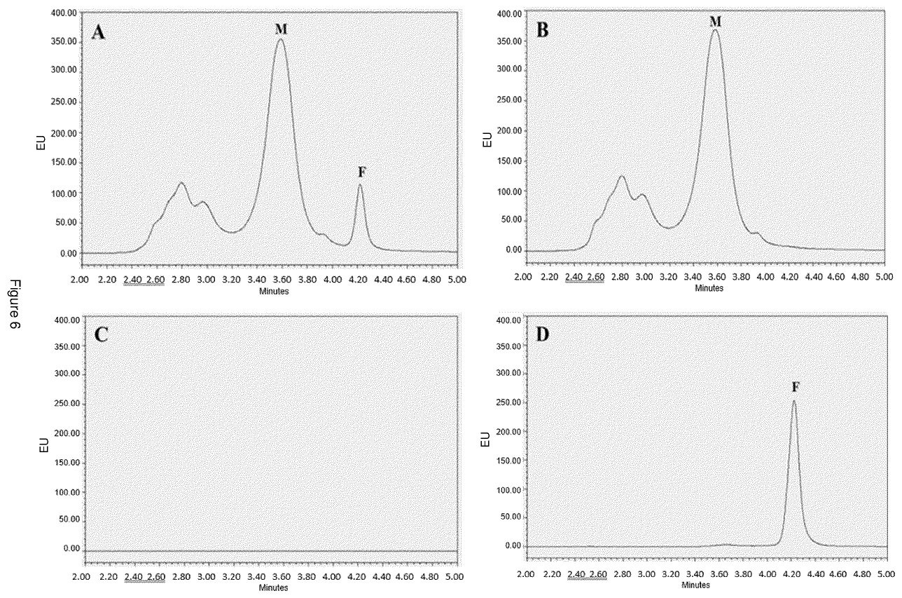

As shown in FIG. 6, analysis by size exclusion chromatography shows only the acidic elution pool (panel A) has a peak corresponding to the Fab (“F”) which is absent from the alkali elution pool (panel B). There is little/no protein in the strip that followed the acidic elution (panel C) as all protein was already eluted by the acidic elution, whereas the strip following the alkali elution (panel D) contains a peak corresponding to the Fab.

The efficient elution of the FST-Fab fusion protein of the invention from Protein A under slightly alkali conditions is a unique property of this molecule. It presents several potential advantages with regards to the downstream process. Firstly, by not using acidic elution conditions it is possible to avoid co-elution of Fab as the Fab remains strongly bound to Protein A. Additionally, the Fst-Fab is not exposed to harsh acidic pH's for a prolonged period. Finally, the alkali elution is compatible with subsequent chromatography steps, meaning reduced sample manipulation and therefore potential increases in yield and recovery.

### Example 6—Production of Stable Cell Lines

All the previous examples were performed with FST-fusion proteins transiently expressed (as per the section material & methods above), this example focuses on the obtention of stable cell lines to produce the FST-fusion proteins according to the invention.

Transfection of Host Cell Line: CHO DG44 (dhfr-) host cells were transfected with DNA double gene vector plasmid for stable expression of human FST315(HBM)-645 Fab molecule (i.e. encoding SEQ ID NO: 8 and SEQ ID NO:6) and a selectable marker dihydrofolate reductase (DHFR). The vector was linearized prior to electroporation. Cells were electroporated and then allowed to recover in a static, temperature and C02 controlled incubator in host cell growth medium for 24h, before being cultured in selective medium.

In total 167 mini-pools were recovered and cultured in selective media containing methotrexate. Based on antibody titre, 70 mini-pools were taken through to evaluation in shake flask cultures. Based on 10-day batch mAb titre, the top 24 mini-pools were selected for assessment in the AMBR automated microscale bioreactors.

The best 7 mini-pools (MP) were selected for single cell cloning. Cells from each MP were centrifuged, and the pellet re-suspended in PBS. Each MP cell suspension was then individually analyzed by flow cytometry. Following single cell cloning the top 54 clones based on the highest antibody titre were expanded to shake flask for batch assessment. 12 high expressing clones were chosen and evaluated in several fed batch processes in AMBR microscale bioreactor system using animal origin-free, chemically-defined medium to evaluate clone cell growth, antibody titre, specific productivity and product quality.

A lead clone was selected, and production runs in Wave Bioreactors and 5 L Shake flasks were carried out using best fed batch process determined previously.

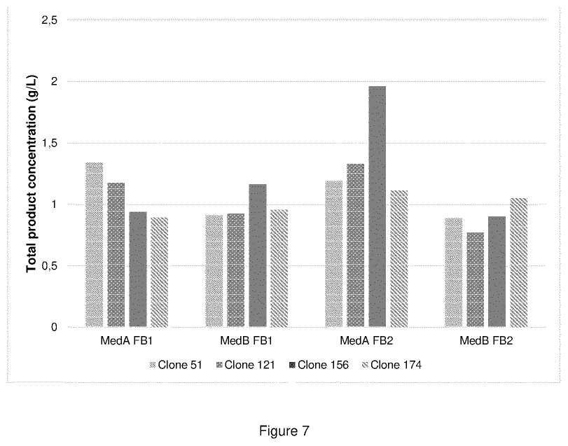

The total product concentration achieved in two different media and fed batch processes from 4 different clones are shown (see FIG. 7). This example shows that an aspect to consider, if one wishes to possibly improve the yield of production of an FST-fusion protein according to the invention, is the set of media (basal medium as well as feed medium/media) to be used for their production. As shown in FIG. 7, although clone 156 was generally produced at about 1 g/L at small scale, the use of basal medium A in combination with fed batch process 2 (FB2) led to a doubling of the titre compared to fed batch process 1 (FB1). As another example, clone 51 was produced at about 1.2-1.3 g/L in the presence of basal medium A (whatever the feed medium/media), but said production dropped below 1 g/L with basal medium B.

TABLE 1
Pharmacokinetic parameters from example 2

| | | | FST315 | | | FST288 |
|---|---|---|---|---|---|---|
| | FST315 | FST315- | HBM- | FST288 | FST288- | HBM- |
| | WT | Fab | Fab | WT | Fab | Fab |
| CL | 162 | 28.6 | 2.98 | 324 | 592 | 34.2 |
| (mL/h/kg) | | | | | | |
| Vₛₛ (L/kg) | 0.26 | 0.25 | 0.039 | 0.78 | ND* | 0.38 |
| T_{1/2} (h) | 4.0 | 16.3 | 23.1 | 3.7 | 7.8 | 27 |
| MRTₗₐₛₜ | 1.6 | 9.3 | 13.1 | 2.3 | 5.2 | 11 |
| (h) | | | | | | |

*Not determined

TABLE 2
Kd binding affinity (from Biacore assay) from example 3
Biacore Kd values (pM)

| | | | | | Human |
|---|---|---|---|---|---|
| Ligands | Activin A | Activin B | GDF8 | GDF11 | Albumin |
| FST315WT-Fab | 200 | 69 | 150 | NA* | NA* |
| FST315HBM-Fab | 11 | 11 | 2300 | 1250 | 2602 |
| FST288WT-Fab | 32 | 71 | 240 | NA* | NA* |
| FST288HBM-Fab | 42 | 50 | 170 | NA* | NA* |

*Not available

TABLE 3
IC50 data from example 4

| | Activin A | | Activin B | | GDF8/Myostatin | | GDF11 | |
|---|---|---|---|---|---|---|---|---|
| Follistatin | IC50 | Range | IC50 | Range | IC50 | Range | IC50 | Range |
| moiety | (nM) | (nM) | (nM) | (nM) | (nM) | (nM) | (nM) | (nM) |
| FST315HBM-Fab | 0.10 | 0.068- | 0.46 | 0.32- | 1.40 | 1.23- | 4.24 | 2.33- |
| (n = 7) | | 0.11 | | 0.62 | | 1.7 | | 12.2 |
| FST315WT | 0.31 | 0.26- | 1.57 | 0.92- | 3.08 | 1.8- | 8.37 | 5.44- |
| (n = 4) | | 0.37 | | 2.16 | | 3.93 | | 18.1 |
| FST288HBM-Fab | 0.14 | 0.13- | 0.50 | 0.3- | 0.45 | 0.32- | 0.65 | 0.51- |
| (n = 4) | | 0.14 | | 1.17 | | 0.5 | | 0.84 |
| FST288WT | 0.14 | 0.13- | 0.45 | 0.22- | 0.73 | 0.59- | 0.77 | 0.64- |
| (n = 4) | | 0.15 | | 1.4 | | 0.82 | | 0.95 |

TABLE 4
Recovery post affinity chromatography of
the different constructs of Example 5.

| | Human VH3 | Recovery post affinity chromatography (%) | |
|---|---|---|---|
| | domain | Protein A | Protein G* |
| FST-Fab 1 | Yes | 80 | 100 |
| FST-Fab 2 | No | — | 91 |
| FST- Fab 3 | No | — | 108 |

** All the data in thisTable were normalised based on the results of the Protein G recovery for FST-Fab1 (considered at 100%)

### REFERENCES

1. WO2015/187977
2. WO2017/152090
3. Sidis et al. (2005) Endocrinology 146(1):130-136
7. http://blast.ncbi.nlm.nih.gov/
8. http://www.ebi.ac.uk/Tools/emboss
9. WO2013/068571
10. “Current Protocols in Molecular Biology” (1999) F. M. Ausubel (ed), Wiley Interscience, New York and the Maniatis Manual produced by Cold Spring Harbor Publishing
11. Charlton, Methods in Molecular Biology, Vol. 248 (B. K. C. Lo, ed., Humana Press, Totowa, NJ, 2003, pp. 245-254, describing expression of antibody fragments in E. coli.)
12. Gerngross et al (2004), Nat. Biotech. 22: 1409-1414
13. Li et al. (2006) Nat. Biotech. 24:210-215.
14. Remington: The Science and Practice of Pharmacy, Alfonso R. Gennaro (Editor) Mack Publishing Company, April 1997
15. Cain et al. (2013) Biotechnol Prog. 29(3): 697-706.
16. Adams et al. (2016) MABS 8(7): 1336-1346.
17. Sidis et al. (2006) Endocrinology 147(7):3586-3597

1. A fusion protein comprising:
a. a follistatin moiety,
b. an antibody moiety, and optionally
c. a linker between the follistatin moiety and the antibody moiety.

2. The fusion protein according to claim 1, wherein the follistatin moiety comprises or is a naturally occurring protein, a functional fragment thereof and/or a functional variant thereof.

3. The fusion protein according to claim 1, wherein the follistatin moiety comprises any one of:
a. SEQ ID NO: 1;
b. SEQ ID NO: 2;
c. SEQ ID NO: 3;
d. SEQ ID NO: 4;
e. any protein comprising amino acid residues comprising between 289 and 314 residues of any one of SEQ ID NO. 1 to 4; or
f. a sequence having at least 95% sequence identity to any one of SEQ ID NOs: 1 to 4.

4. The fusion protein according to claim 1, wherein the antibody moiety binds albumin.

5. The fusion protein according to claim 1, wherein the antibody moiety is a chimeric, humanized or human antibody moiety.

6. The fusion protein according to claim 1, wherein the antibody moiety is:
a) an antigen-binding moiety selected from a Fab, a Fab′, or a F(ab′)2 or b) an antigen-binding moiety selected from a Fab, a Fab′, or a F(ab′)2 and further comprising a human VH3 domain which is capable of binding protein A

7. The fusion protein according to claim 1, wherein the antibody moiety comprises:
a. a light chain variable region comprising at least one CDR selected from: a CDR-L1 comprising SEQ ID NO: 13; a CDR-L2 comprising SEQ ID NO: 14 and/or a CDR-L3 comprising SEQ ID NO: 15; and a heavy chain variable region comprising at least one CDR selected from a CDR-H1 comprising SEQ ID NO: 16; a CDR-H2 comprising SEQ ID NO: 17 and/or a CDR-H3 comprising SEQ ID NO: 18; or
b. a heavy chain variable region comprising of SEQ ID NO: 5, or a sequence having at least 95% identity thereto and a light chain variable region comprising or consisting of SEQ ID NO: 6, or a sequence having at least 95% identity thereto.

8. The fusion protein according to any claim 1, wherein the fusion protein comprises a linker between the follistatin moiety and the antibody moiety and wherein the linker is selected from the group consisting of: SGGGGS (SEQ ID NO: 7), SGGGGSSGGGGS (SEQ ID NO: 19), GGGGS (SEQ ID NO: 20) and GGGGSGGGGS (SEQ ID NO: 21).

9. The fusion protein according to claim 1, wherein:
a) the antibody moiety is connected to the C-terminal portion of the follistatin moiety, or
b) if a linker is present, the antibody moiety is connected to the C-terminal portion of the follistatin moiety by the linker.

10. The fusion protein according to claim 1, wherein the fusion protein comprises:
(a) i. an FST315 polypeptide defined by SEQ ID NO: 1, or a sequence having at least 95% sequence identity thereto;
ii. a Fab heavy chain defined by SEQ ID NO: 5, or a sequence having at least 95% sequence identity thereto, conjugated to the C-terminus of the FST315 polypeptide; and
iii. a Fab light chain defined by SEQ ID NO: 6, or a sequence having at least 95% sequence identity thereto;
(b) i. an FST288 polypeptide defined by SEQ ID NO: 2, or a sequence having at least 95% sequence identity thereto;
ii. a Fab heavy chain defined by SEQ ID NO: 5, or a sequence having at least 95% sequence identity thereto, conjugated to the C-terminus of the FST288 polypeptide; and
iii. a Fab light chain defined by SEQ ID NO: 6, or a sequence having at least 95% sequence identity thereto;
(c) i. an FST315HBM polypeptide defined by SEQ ID NO: 3, or a sequence having at least 95% sequence identity thereto;
ii. a Fab heavy chain defined by SEQ ID NO: 5, or a sequence having at least 95% sequence identity thereto, conjugated to the C-terminus of the FST315HBM polypeptide; and
iii. a Fab light chain defined by SEQ ID NO: 6, or a sequence having at least 95% sequence identity thereto;
(d) i. an FST288HBM polypeptide defined by SEQ ID NO: 4, or a sequence having at least 95% sequence identity thereto;
ii. a Fab heavy chain defined by SEQ ID NO: 5, or a sequence having at least 95% sequence identity thereto, conjugated to the C-terminus of the FST288HBM polypeptide; and
iii. a Fab light chain defined by SEQ ID NO: 6, or a sequence having at least 95% sequence identity thereto;
(e) i. an FST315 polypeptide defined by SEQ ID NO: 1, or a sequence having at least 95% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide;
iii. a Fab heavy chain defined by SEQ ID NO: 5, or a sequence having at least 95% sequence identity thereto conjugated to the free-terminus of the linker; and
iv. a Fab light chain defined by SEQ ID NO: 6, or a sequence having at least 95% sequence identity thereto;
(f) i. an FST288 polypeptide defined by SEQ ID NO: 2, or a sequence having at least 95% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide;
iii. a Fab heavy chain defined by SEQ ID NO: 5, or a sequence having at least 95% sequence identity thereto conjugated to the free-terminus of the linker; and
iv. a Fab light chain defined by SEQ ID NO: 6, or a sequence having at least 95% sequence identity thereto;
(g) i. an FST315 polypeptide variant defined by SEQ ID NO: 3 (FST315HBM) or SEQ ID NO: 22, or a sequence having at least 95%, identity thereto;
ii. a linker defined by SEQ ID NO: 7, SEQ ID NO: 19, SEQ ID NO: 20 or SEQ ID NO: 21 conjugated to the C-terminus of the FST315 polypeptide variant;
iii. a Fab heavy chain defined by SEQ ID NO: 5, or a sequence having at least 95% sequence identity thereto conjugated to the free-terminus of the linker; and
iv. a Fab light chain defined by SEQ ID NO: 6, or a sequence having at least 95% sequence identity thereto;
(h) i. an FST288 polypeptide variant defined by SEQ ID NO: 4 (FST288HBM) or SEQ ID NO:25, or a sequence having at least 95% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide variant;
iii. a Fab heavy chain defined by SEQ ID NO: 5, or a sequence having at least 95% sequence identity thereto conjugated to the free-terminus of the linker; and
iv. a Fab light chain defined by SEQ ID NO: 6, or a sequence having at least 95% sequence identity thereto;
(i) SEQ ID NO: 8, 9, 10, 11, 24, 25, 26, 27, 32, 33, 34 or 35 or a sequence having at least 95% sequence identity thereto and a Fab light chain defined by SEQ ID NO: 6, or a sequence having at least 95% sequence identity thereto;
(j) SEQ ID NO: 8, 9, 10, 11, 24, 25, 26, 27, 32, 33, 34 or 35 and a Fab light chain defined by SEQ ID NO: 6; or
(k) a functional variant or fragment of any one of (a) to (j).

11. An isolated polynucleotide encoding the fusion proteins according to claim 1.

12. A cloning or expression vector comprising one or more polynucleotides according to claim 11.

13. A host cell comprising:
one or more polynucleotide(s) according to claim 11.

14. A process for the production of a fusion protein according to claim 1, comprising culturing a host cell comprising a polynucleotide encoding the fusion protein of claim 1 or an expression vector comprising a polynucleotide encoding the fusion protein of claim 1 under suitable conditions for producing the fusion protein and isolating the fusion protein.

15. A pharmaceutical composition comprising the fusion protein according to claim 1 and one or more pharmaceutically acceptable carriers, excipients or diluents.

16. A method of treating a subject in need thereof, the method comprising administering the subject the fusion protein according to claim 1.


SEQUENCE LISTING (66 sequences)

SEQ ID NO: 1 - protein, 315 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 2 - protein, 288 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 3 - protein, 315 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 4 - protein, 288 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 5 - protein, 224 aa, synthetic construct
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 217 aa, synthetic construct
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 6 aa, synthetic construct
SGGGGS

SEQ ID NO: 8 - protein, 545 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 539 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 518 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 11 - protein, 512 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - protein, 29 aa, synthetic construct
MVRARHQPGGLCLLLLLLCQFMEDRSAQA

SEQ ID NO: 13 - protein, 12 aa, synthetic construct
QSSPSVWSNFLS

SEQ ID NO: 14 - protein, 7 aa, synthetic construct
EASKLTS

SEQ ID NO: 15 - protein, 11 aa, synthetic construct
GGGYSSISDTT

SEQ ID NO: 16 - protein, 10 aa, synthetic construct
GIDLSNYAIN

SEQ ID NO: 17 - protein, 16 aa, synthetic construct
IIWASGTTFYATWAKG

SEQ ID NO: 18 - protein, 13 aa, synthetic construct
TVPGYSTAPYFDL

SEQ ID NO: 19 - protein, 12 aa, synthetic construct
SGGGGSSGGGGS

SEQ ID NO: 20 - protein, 5 aa, synthetic construct
GGGGS

SEQ ID NO: 21 - protein, 10 aa, synthetic construct
GGGGSGGGGS

SEQ ID NO: 22 - protein, 315 aa, synthetic construct
  VARIANT 76: note = any amino acids
  VARIANT 81..82: note = any amino acids
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 23 - protein, 288 aa, synthetic construct
  VARIANT 76: note = any amino acids
  VARIANT 81..82: note = any amino acids
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 24 - protein, 539 aa, synthetic construct
  VARIANT 76: note = any amino acids
  VARIANT 81..82: note = any amino acids
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - protein, 545 aa, synthetic construct
  VARIANT 76: note = any amino acids
  VARIANT 81..82: note = any amino acids
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 512 aa, synthetic construct
  VARIANT 76: note = any amino acids
  VARIANT 81..82: note = any amino acids
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 518 aa, synthetic construct
  VARIANT 76: note = any amino acids
  VARIANT 81..82: note = any amino acids
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 539 aa, synthetic construct
  VARIANT 300: note = any amino acids
  VARIANT 305..306: note = any amino acids
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 29 - protein, 545 aa, synthetic construct
  VARIANT 306: note = any amino acids
  VARIANT 311..312: note = any amino acids
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 30 - protein, 512 aa, synthetic construct
  VARIANT 300: note = any amino acids
  VARIANT 305..306: note = any amino acids
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 31 - protein, 518 aa, synthetic construct
  VARIANT 306: note = any amino acids
  VARIANT 311..312: note = any amino acids
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 32 - protein, 545 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 539 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 34 - protein, 518 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 35 - protein, 512 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - DNA, 945 bp, Homo sapiens
  source 1..945: mol_type = other DNA
gggaactgttggctgagacaggccaagaacggacgctgccaggtcctgtacaagactgaattgtccaaggaagagtgctgctccaccggacggctgtcaaccagctggactgaagaggacgtgaacgacaacaccctcttcaagtggatgatcttcaacggtggcgcacctaactgtattccgtgtaaagaaacctgtgaaaacgtcgactgtggacccgggaagaagtgcaggatgaacaagaagaacaagccgcgctgcgtgtgtgcacctgactgctcgaacatcacctggaagggcccagtctgtgggctggacggaaagacctaccggaacgaatgtgccctgctgaaggctcggtgcaaggagcaaccggaactggaagtgcagtaccaggggcggtgcaagaaaacctgtcgggatgtgttttgccctggatcctccacttgcgtggtggaccagaccaacaacgcctactgtgtgacatgcaacaggatctgcccggagcctgcttcctcggagcagtacctctgcggcaatgatggcgtgacctactcctccgcctgtcacttgcgcaaagccacttgcctgctgggacgcagcatcgggctcgcctatgagggaaagtgtattaaggccaagtcctgcgaggacatccagtgcaccggcggaaagaagtgcctctgggatttcaaagttggccggggcagatgctcgctgtgcgatgagctgtgcccggactcgaagtcggacgaacccgtctgtgctagcgataacgcgacctacgcatcggagtgcgccatgaaggaggccgcatgctctagcggagtgctgctggaggtcaagcactccggttcctgcaactccatcagcgaagataccgaagaagaagaggaagatgaggaccaggactattccttcccgatctcatccattctggaatgg

SEQ ID NO: 37 - DNA, 864 bp, synthetic construct
  source 1..864: mol_type = other DNA
gggaactgttggctgagacaggccaagaacggacgctgccaggtcctgtacaagactgaattgtccaaggaagagtgctgctccaccggacggctgtcaaccagctggactgaagaggacgtgaacgacaacaccctcttcaagtggatgatcttcaacggtggcgcacctaactgtattccgtgtaaagaaacctgtgaaaacgtcgactgtggacccgggaagaagtgcaggatgaacaagaagaacaagccgcgctgcgtgtgtgcacctgactgctcgaacatcacctggaagggcccagtctgtgggctggacggaaagacctaccggaacgaatgtgccctgctgaaggctcggtgcaaggagcaaccggaactggaagtgcagtaccaggggcggtgcaagaaaacctgtcgggatgtgttttgccctggatcctccacttgcgtggtggaccagaccaacaacgcctactgtgtgacatgcaacaggatctgcccggagcctgcttcctcggagcagtacctctgcggcaatgatggcgtgacctactcctccgcctgtcacttgcgcaaagccacttgcctgctgggacgcagcatcgggctcgcctatgagggaaagtgtattaaggccaagtcctgcgaggacatccagtgcaccggcggaaagaagtgcctctgggatttcaaagttggccggggcagatgctcgctgtgcgatgagctgtgcccggactcgaagtcggacgaacccgtctgtgctagcgataacgcgacctacgcatcggagtgcgccatgaaggaggccgcatgctctagcggagtgctgctggaggtcaagcactccggttcctgcaac

SEQ ID NO: 38 - DNA, 945 bp, synthetic construct
  source 1..945: mol_type = other DNA
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaaggcctgccggatgaacgcggccaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaatagcatctccgaggacactgaggaggaagaggaagatgaagatcaggactacagctttccgatttcctcaatcttggagtgg

SEQ ID NO: 39 - DNA, 864 bp, synthetic construct
  source 1..864: mol_type = other DNA
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaaggcctgccggatgaacgcggccaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaat

SEQ ID NO: 40 - DNA, 672 bp, synthetic construct
  source 1..672: mol_type = other DNA
gaggttcagctgctggagtctggaggcgggcttgtccagcctggagggagcctgcgtctctcttgtgcagtaagcggcatcgacctgtccaactacgcgattaactgggtacgtcaggcaccgggtaaaggtctggaatggatcggcatcatctgggcctctggtacgaccttctacgctacttgggccaaaggtcgtttcaccatctcccgtgacaatagcaaaaacaccgtgtacctgcagatgaactctctgcgtgcggaagacactgcggtttactattgcgcgcgtaccgttccgggctattctactgcaccgtacttcgacctgtggggtcagggtactctggttaccgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 41 - DNA, 651 bp, synthetic construct
  source 1..651: mol_type = other DNA
gatatccagatgacccagagtccaagcagtgtttccgccagcgtaggcgatcgtgtgactattacctgtcagtcctctccgagcgtttggtccaacttcctgagctggtaccagcagaaaccgggtaaagccccgaaactgctgatctacgaggcgtctaaactgacctctggtgtaccgtcccgtttcagcggctctggctctggtacggacttcactctgaccatctcctctctgcagccggaagactttgcaacgtactactgcggtggtggttactcttccatctctgacaccacgttcggtggtggcaccaaagttgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - DNA, 18 bp, synthetic construct
  source 1..18: mol_type = other DNA
tcgggtggaggaggctca

SEQ ID NO: 43 - DNA, 1635 bp, synthetic construct
  source 1..1635: mol_type = other DNA
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaaggcctgccggatgaacgcggccaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaatagcatctccgaggacactgaggaggaagaggaagatgaagatcaggactacagctttccgatttcctcaatcttggagtggtccggaggaggtggaagcgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 44 - DNA, 1617 bp, synthetic construct
  source 1..1617: mol_type = other DNA
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaaggcctgccggatgaacgcggccaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaatagcatctccgaggacactgaggaggaagaggaagatgaagatcaggactacagctttccgatttcctcaatcttggagtgggaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 45 - DNA, 1554 bp, synthetic construct
  source 1..1554: mol_type = other DNA
ggaaactgctggctgagacaggccaagaacggccgctgtcaagtgctgtacaagaccgagttgtccaaggaggaatgttgtagcaccggccggttgtccacctcctggaccgaggaggacgtgaacgataacaccctgttcaagtggatgattttcaacggcggtgccccaaattgcatcccctgcaaggagacttgcgaaaatgtggactgcggaccgggaaaggcctgtcggatgaacgctgcgaacaagccgcgctgcgtgtgtgccccggactgctccaacatcacttggaagggccccgtgtgtggactggacggaaagacctaccgcaacgagtgcgccctgctgaaggcccggtgcaaggagcaacctgagttagaagtgcaataccagggccggtgcaaaaagacttgccgcgatgtgttctgtcccgggtccagcacttgcgtggtggatcagaccaacaacgcctactgcgtgacgtgcaaccggatttgcccagagccggcatcttccgaacaatacctgtgcggcaacgatggcgtgacctactcgtccgcctgccatctccggaaggccacttgtctgctggggaggtcgattggactcgcatacgaagggaagtgcattaaggctaagtcctgcgaggacatccagtgcaccggcggaaagaagtgcctgtgggacttcaaagtcggaaggggtcggtgttccctgtgcgacgaactgtgtcccgactccaagtcggacgaacctgtgtgcgcgagcgataacgctacatacgcctcggaatgcgccatgaaagaggcggcttgctcatccggcgtcctgctggaagtgaagcattccggaagctgcaattccggaggaggtggaagcgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 46 - DNA, 1536 bp, synthetic construct
  source 1..1536: mol_type = other DNA
ggaaactgctggctgagacaggccaagaacggccgctgtcaagtgctgtacaagaccgagttgtccaaggaggaatgttgtagcaccggccggttgtccacctcctggaccgaggaggacgtgaacgataacaccctgttcaagtggatgattttcaacggcggtgccccaaattgcatcccctgcaaggagacttgcgaaaatgtggactgcggaccgggaaaggcctgtcggatgaacgctgcgaacaagccgcgctgcgtgtgtgccccggactgctccaacatcacttggaagggccccgtgtgtggactggacggaaagacctaccgcaacgagtgcgccctgctgaaggcccggtgcaaggagcaacctgagttagaagtgcaataccagggccggtgcaaaaagacttgccgcgatgtgttctgtcccgggtccagcacttgcgtggtggatcagaccaacaacgcctactgcgtgacgtgcaaccggatttgcccagagccggcatcttccgaacaatacctgtgcggcaacgatggcgtgacctactcgtccgcctgccatctccggaaggccacttgtctgctggggaggtcgattggactcgcatacgaagggaagtgcattaaggctaagtcctgcgaggacatccagtgcaccggcggaaagaagtgcctgtgggacttcaaagtcggaaggggtcggtgttccctgtgcgacgaactgtgtcccgactccaagtcggacgaacctgtgtgcgcgagcgataacgctacatacgcctcggaatgcgccatgaaagaggcggcttgctcatccggcgtcctgctggaagtgaagcattccggaagctgcaatgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 47 - DNA, 87 bp, synthetic construct
  source 1..87: mol_type = other DNA
atggtccgggccagacaccagccgggaggtctttgtctgctgcttctgctgctctgccaattcatggaggatcggtccgcccaagct

SEQ ID NO: 48 - DNA, 36 bp, synthetic construct
  source 1..36: mol_type = other DNA
cagtcctctccgagcgtttggtccaacttcctgagc

SEQ ID NO: 49 - DNA, 21 bp, synthetic construct
  source 1..21: mol_type = other DNA
gaggcgtctaaactgacctct

SEQ ID NO: 50 - DNA, 33 bp, synthetic construct
  source 1..33: mol_type = other DNA
ggtggtggttactcttccatctctgacaccacg

SEQ ID NO: 51 - DNA, 30 bp, synthetic construct
  source 1..30: mol_type = other DNA
ggcatcgacctgtccaactacgcgattaac

SEQ ID NO: 52 - DNA, 48 bp, synthetic construct
  source 1..48: mol_type = other DNA
atcatctgggcctctggtacgaccttctacgctacttgggccaaaggt

SEQ ID NO: 53 - DNA, 39 bp, synthetic construct
  source 1..39: mol_type = other DNA
accgttccgggctattctactgcaccgtacttcgacctg

SEQ ID NO: 54 - DNA, 36 bp, synthetic construct
  source 1..36: mol_type = other DNA
tcgggtggaggaggctcatcgggtggaggaggctca

SEQ ID NO: 55 - DNA, 15 bp, synthetic construct
  source 1..15: mol_type = other DNA
ggtggaggaggctca

SEQ ID NO: 56 - DNA, 30 bp, synthetic construct
  source 1..30: mol_type = other DNA
ggtggaggaggctcaggtggaggaggctca

SEQ ID NO: 57 - DNA, 945 bp, synthetic construct
  source 1..945: mol_type = other DNA
  misc_difference 226..228: note = n is a, c, g, or t
  misc_difference 241..246: note = n is a, c, g, or t
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaagnnntgccggatgaacnnnnnnaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaatagcatctccgaggacactgaggaggaagaggaagatgaagatcaggactacagctttccgatttcctcaatcttggagtgg

SEQ ID NO: 58 - DNA, 864 bp, synthetic construct
  source 1..864: mol_type = other DNA
  misc_difference 226..228: note = n is a, c, g, or t
  misc_difference 241..246: note = n is a, c, g, or t
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaagnnntgccggatgaacnnnnnnaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaat

SEQ ID NO: 59 - DNA, 1617 bp, synthetic construct
  source 1..1617: mol_type = other DNA
  misc_difference 226..228: note = n is a, c, g, or t
  misc_difference 241..246: note = n is a, c, g, or t
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaagnnntgccggatgaacnnnnnnaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaatagcatctccgaggacactgaggaggaagaggaagatgaagatcaggactacagctttccgatttcctcaatcttggagtgggaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 60 - DNA, 1635 bp, synthetic construct
  source 1..1635: mol_type = other DNA
  misc_difference 226..228: note = n is a, c, g, or t
  misc_difference 241..246: note = n is a, c, g, or t
gggaactgttggctgagacaggccaagaacgggcgctgccaagtgttgtataagaccgagctgtcgaaggaggaatgctgcagcactggccggctttcaacctcctggaccgaagaagatgtcaacgataacaccctgttcaagtggatgatcttcaacggtggagcccccaactgcatcccgtgtaaagaaacttgcgaaaatgtggattgtggccccgggaagnnntgccggatgaacnnnnnnaacaagccgagatgcgtgtgtgcccccgactgctcgaacatcacctggaagggacctgtgtgcggcctggacggaaagacctaccggaatgagtgcgccctgctgaaggcccggtgtaaagaacagcctgagttggaagtgcagtaccagggtcgctgcaagaaaacctgtcgggacgtgttctgtccgggctcctcaacttgcgtggtggaccagactaacaacgcctactgcgtgacttgcaacaggatctgcccagagccggcttcgtccgagcaatacctgtgcggaaacgacggcgtgacctactccagcgcgtgccatctccggaaggccacatgcctgctcggacggtccattggactggcctacgagggaaagtgcatcaaggctaagtcctgcgaagatattcagtgcaccggcggaaagaagtgcctctgggacttcaaagtcggtagagggcgctgctctctgtgcgacgaactgtgcccagattccaagtccgacgagcctgtctgtgcctccgacaacgcaacctacgcctccgaatgcgccatgaaggaagccgcgtgctcatccggagtgctgcttgaagtcaagcactcgggctcgtgtaatagcatctccgaggacactgaggaggaagaggaagatgaagatcaggactacagctttccgatttcctcaatcttggagtggtccggaggaggtggaagcgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 61 - DNA, 1536 bp, synthetic construct
  source 1..1536: mol_type = other DNA
  misc_difference 226..228: note = n is a, c, g, or t
  misc_difference 241..246: note = n is a, c, g, or t
ggaaactgctggctgagacaggccaagaacggccgctgtcaagtgctgtacaagaccgagttgtccaaggaggaatgttgtagcaccggccggttgtccacctcctggaccgaggaggacgtgaacgataacaccctgttcaagtggatgattttcaacggcggtgccccaaattgcatcccctgcaaggagacttgcgaaaatgtggactgcggaccgggaaagnnntgtcggatgaacnnnnnnaacaagccgcgctgcgtgtgtgccccggactgctccaacatcacttggaagggccccgtgtgtggactggacggaaagacctaccgcaacgagtgcgccctgctgaaggcccggtgcaaggagcaacctgagttagaagtgcaataccagggccggtgcaaaaagacttgccgcgatgtgttctgtcccgggtccagcacttgcgtggtggatcagaccaacaacgcctactgcgtgacgtgcaaccggatttgcccagagccggcatcttccgaacaatacctgtgcggcaacgatggcgtgacctactcgtccgcctgccatctccggaaggccacttgtctgctggggaggtcgattggactcgcatacgaagggaagtgcattaaggctaagtcctgcgaggacatccagtgcaccggcggaaagaagtgcctgtgggacttcaaagtcggaaggggtcggtgttccctgtgcgacgaactgtgtcccgactccaagtcggacgaacctgtgtgcgcgagcgataacgctacatacgcctcggaatgcgccatgaaagaggcggcttgctcatccggcgtcctgctggaagtgaagcattccggaagctgcaatgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 62 - DNA, 1554 bp, synthetic construct
  source 1..1554: mol_type = other DNA
  misc_difference 226..228: note = n is a, c, g, or t
  misc_difference 241..246: note = n is a, c, g, or t
ggaaactgctggctgagacaggccaagaacggccgctgtcaagtgctgtacaagaccgagttgtccaaggaggaatgttgtagcaccggccggttgtccacctcctggaccgaggaggacgtgaacgataacaccctgttcaagtggatgattttcaacggcggtgccccaaattgcatcccctgcaaggagacttgcgaaaatgtggactgcggaccgggaaagnnntgtcggatgaacnnnnnnaacaagccgcgctgcgtgtgtgccccggactgctccaacatcacttggaagggccccgtgtgtggactggacggaaagacctaccgcaacgagtgcgccctgctgaaggcccggtgcaaggagcaacctgagttagaagtgcaataccagggccggtgcaaaaagacttgccgcgatgtgttctgtcccgggtccagcacttgcgtggtggatcagaccaacaacgcctactgcgtgacgtgcaaccggatttgcccagagccggcatcttccgaacaatacctgtgcggcaacgatggcgtgacctactcgtccgcctgccatctccggaaggccacttgtctgctggggaggtcgattggactcgcatacgaagggaagtgcattaaggctaagtcctgcgaggacatccagtgcaccggcggaaagaagtgcctgtgggacttcaaagtcggaaggggtcggtgttccctgtgcgacgaactgtgtcccgactccaagtcggacgaacctgtgtgcgcgagcgataacgctacatacgcctcggaatgcgccatgaaagaggcggcttgctcatccggcgtcctgctggaagtgaagcattccggaagctgcaattccggaggaggtggaagcgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 63 - DNA, 1635 bp, synthetic construct
  source 1..1635: mol_type = other DNA
gggaactgttggctgagacaggccaagaacggacgctgccaggtcctgtacaagactgaattgtccaaggaagagtgctgctccaccggacggctgtcaaccagctggactgaagaggacgtgaacgacaacaccctcttcaagtggatgatcttcaacggtggcgcacctaactgtattccgtgtaaagaaacctgtgaaaacgtcgactgtggacccgggaagaagtgcaggatgaacaagaagaacaagccgcgctgcgtgtgtgcacctgactgctcgaacatcacctggaagggcccagtctgtgggctggacggaaagacctaccggaacgaatgtgccctgctgaaggctcggtgcaaggagcaaccggaactggaagtgcagtaccaggggcggtgcaagaaaacctgtcgggatgtgttttgccctggatcctccacttgcgtggtggaccagaccaacaacgcctactgtgtgacatgcaacaggatctgcccggagcctgcttcctcggagcagtacctctgcggcaatgatggcgtgacctactcctccgcctgtcacttgcgcaaagccacttgcctgctgggacgcagcatcgggctcgcctatgagggaaagtgtattaaggccaagtcctgcgaggacatccagtgcaccggcggaaagaagtgcctctgggatttcaaagttggccggggcagatgctcgctgtgcgatgagctgtgcccggactcgaagtcggacgaacccgtctgtgctagcgataacgcgacctacgcatcggagtgcgccatgaaggaggccgcatgctctagcggagtgctgctggaggtcaagcactccggttcctgcaactccatcagcgaagataccgaagaagaagaggaagatgaggaccaggactattccttcccgatctcatccattctggaatggtccggaggaggtggaagcgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 64 - DNA, 1617 bp, synthetic construct
  source 1..1617: mol_type = other DNA
gggaactgttggctgagacaggccaagaacggacgctgccaggtcctgtacaagactgaattgtccaaggaagagtgctgctccaccggacggctgtcaaccagctggactgaagaggacgtgaacgacaacaccctcttcaagtggatgatcttcaacggtggcgcacctaactgtattccgtgtaaagaaacctgtgaaaacgtcgactgtggacccgggaagaagtgcaggatgaacaagaagaacaagccgcgctgcgtgtgtgcacctgactgctcgaacatcacctggaagggcccagtctgtgggctggacggaaagacctaccggaacgaatgtgccctgctgaaggctcggtgcaaggagcaaccggaactggaagtgcagtaccaggggcggtgcaagaaaacctgtcgggatgtgttttgccctggatcctccacttgcgtggtggaccagaccaacaacgcctactgtgtgacatgcaacaggatctgcccggagcctgcttcctcggagcagtacctctgcggcaatgatggcgtgacctactcctccgcctgtcacttgcgcaaagccacttgcctgctgggacgcagcatcgggctcgcctatgagggaaagtgtattaaggccaagtcctgcgaggacatccagtgcaccggcggaaagaagtgcctctgggatttcaaagttggccggggcagatgctcgctgtgcgatgagctgtgcccggactcgaagtcggacgaacccgtctgtgctagcgataacgcgacctacgcatcggagtgcgccatgaaggaggccgcatgctctagcggagtgctgctggaggtcaagcactccggttcctgcaactccatcagcgaagataccgaagaagaagaggaagatgaggaccaggactattccttcccgatctcatccattctggaatgggaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 65 - DNA, 1554 bp, synthetic construct
  source 1..1554: mol_type = other DNA
gggaactgttggctgagacaggccaagaacggacgctgccaggtcctgtacaagactgaattgtccaaggaagagtgctgctccaccggacggctgtcaaccagctggactgaagaggacgtgaacgacaacaccctcttcaagtggatgatcttcaacggtggcgcacctaactgtattccgtgtaaagaaacctgtgaaaacgtcgactgtggacccgggaagaagtgcaggatgaacaagaagaacaagccgcgctgcgtgtgtgcacctgactgctcgaacatcacctggaagggcccagtctgtgggctggacggaaagacctaccggaacgaatgtgccctgctgaaggctcggtgcaaggagcaaccggaactggaagtgcagtaccaggggcggtgcaagaaaacctgtcgggatgtgttttgccctggatcctccacttgcgtggtggaccagaccaacaacgcctactgtgtgacatgcaacaggatctgcccggagcctgcttcctcggagcagtacctctgcggcaatgatggcgtgacctactcctccgcctgtcacttgcgcaaagccacttgcctgctgggacgcagcatcgggctcgcctatgagggaaagtgtattaaggccaagtcctgcgaggacatccagtgcaccggcggaaagaagtgcctctgggatttcaaagttggccggggcagatgctcgctgtgcgatgagctgtgcccggactcgaagtcggacgaacccgtctgtgctagcgataacgcgacctacgcatcggagtgcgccatgaaggaggccgcatgctctagcggagtgctgctggaggtcaagcactccggttcctgcaactccggaggaggtggaagcgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc

SEQ ID NO: 66 - DNA, 1536 bp, synthetic construct
  source 1..1536: mol_type = other DNA
gggaactgttggctgagacaggccaagaacggacgctgccaggtcctgtacaagactgaattgtccaaggaagagtgctgctccaccggacggctgtcaaccagctggactgaagaggacgtgaacgacaacaccctcttcaagtggatgatcttcaacggtggcgcacctaactgtattccgtgtaaagaaacctgtgaaaacgtcgactgtggacccgggaagaagtgcaggatgaacaagaagaacaagccgcgctgcgtgtgtgcacctgactgctcgaacatcacctggaagggcccagtctgtgggctggacggaaagacctaccggaacgaatgtgccctgctgaaggctcggtgcaaggagcaaccggaactggaagtgcagtaccaggggcggtgcaagaaaacctgtcgggatgtgttttgccctggatcctccacttgcgtggtggaccagaccaacaacgcctactgtgtgacatgcaacaggatctgcccggagcctgcttcctcggagcagtacctctgcggcaatgatggcgtgacctactcctccgcctgtcacttgcgcaaagccacttgcctgctgggacgcagcatcgggctcgcctatgagggaaagtgtattaaggccaagtcctgcgaggacatccagtgcaccggcggaaagaagtgcctctgggatttcaaagttggccggggcagatgctcgctgtgcgatgagctgtgcccggactcgaagtcggacgaacccgtctgtgctagcgataacgcgacctacgcatcggagtgcgccatgaaggaggccgcatgctctagcggagtgctgctggaggtcaagcactccggttcctgcaacgaagtccagctgctggaatcaggcggagggcttgtgcagcctgggggaagccttcggttgtcatgtgccgtgtccggtatcgacctgtccaactacgcaatcaactgggttagacaggcgccaggaaagggactggagtggattggcatcatctgggccagcgggactacgttctatgccacctgggccaagggtcgcttcaccatctcgcgggacaactcaaagaacacggtgtacctccagatgaactccctgagagctgaggacactgccgtgtattactgcgcgagaaccgtacctggctactccactgccccctacttcgacctttggggacaggggaccctggtcacagtgtccagcgcctcaaccaagggccccagcgtctttccgcttgccccgagctcgaagtcgaccagcggaggcactgcagccctgggttgtctggtcaaggactacttccctgagcccgtgaccgtgtcatggaacagcggcgccctcacttcgggcgtgcacacttttcctgcggtgctccagtcgtccgggctctactccctgtcatccgtggtcaccgtgccgtcctcgtcactcggaacccagacctacatctgcaacgtgaaccacaagccttccaacaccaaggtcgacaagaaggtcgaacccaagtcctgc